(19) United States
(12) Patent Application Publication
(10) Pub. No.: US 2014/0329854 A1
(43) Pub. Date: Nov. 6, 2014

(54) NOVEL HETEROCYCLIC COMPOUNDS USEFUL IN SIRTUIN BINDING AND MODULATION

(71) Applicant: SMB Innovation, Christiansted, VI (US)

(72) Inventors: Bjarne Due Larsen, Roskilde (DK); Soren Andersen, Christiansted, VI (US); Marianne Spanget Larsen, Ishoj (DK)

(73) Assignee: SMB Innovation, Christiansted, VI (US)

(21) Appl. No.: 14/362,450

(22) PCT Filed: Dec. 12, 2012
(86) PCT No.: PCT/US2012/069151

§ 371 Date: Jun. 3, 2014

Related U.S. Application Data
(60) Provisional application No. 61/569,467, filed on Dec. 12, 2011.

(30) Foreign Application Priority Data
Aug. 1, 2012 (DK) PA 2012 00485

(51) Int. Cl. C07D 215/48; C07D 207/22; C07D 213/81; C07D 213/75; C07D 213/82

(52) U.S. Cl. CPC C07D 215/48; C07D 213/75; C07D 213/82; C07D 213/81; C07D 207/22
USPC 514/311; 514/352; 514/354; 514/355; 514/423; 546/169; 546/309; 546/316; 546/323; 548/537

(57) ABSTRACT

Provided are novel heterocyclic sirtuin binding agents, e.g., N-aryl substituted pyridine dicarboxamides, which are useful as sirtuin modulators. Also provided are methods of using the pyridine dicarboxamide compounds for treating sirtuin mediated disorders and conditions, such as diabetes, cancers, and obesity and diseases related to aging.

### CROSS REFERENCE TO RELATED APPLICATIONS

This application claims the benefit of U.S. Provisional Application No. 61/569,467, filed Dec. 12, 2011, the entirety of which is incorporated herein by reference.

### FIELD OF THE INVENTION

The present invention relates to the preparation of novel heterocyclic compounds and more specifically substituted pyridine dicarboxamides useful in sirtuin binding, activation and modulation. Thus, the invention relates to novel compounds, methods of preparing said compounds, as well as useful intermediates in the methods. The novel compounds of the invention have utility in pharmaceutical formulations for treating diseases such as diabetes, cancers, and obesity and diseases related to ageing.

### BACKGROUND

Silent information regulator 2 (Sir2) proteins, or sirtuins, which comprise a family of enzymes that catalyze the deacetylation of acetyllysine side chains in a reaction that consumes NAD (or NAD+) while simultaneously producing the nicotinamide inhibitor, cf. Avalos et al. (2005). Although several crystal structures of sirtuins bound to non-native acetyl peptides have been determined, relatively little about how sirtuins discriminate among different substrates is understood. Cosgrove, M. S. (2006) has carried out a systematic structural and thermodynamic analysis of several peptides bound to a single sirtuin, the Sir2 homologue from Thermatoga maritima (Sir2Tm)). The sirtuins are encoded by the SIR2 gene family, e.g. the human SIRT1-SIRT7 genes. In eucaryotes, sirtuins regulate transcriptional repression, recombination, the cell division cycle, microtubule organization, and cellular responses to DNA-damaging agents. Sirtuins have also been implicated in regulating the molecular mechanisms of aging. The Sir2 catalytic domain, which is shared among all sirtuins, consists of two distinct domains that bind NAD and the acetyl-lysine substrate, respectively. In addition to the catalytic domain, eukaryotic sirtuins contain variable amino- and carboxy-terminal extensions that regulate their subcellular localizations and catalytic activity. Moreover, the sirtuin proteins are involved in diverse processes from regulation of gene silencing to DNA repair. The Sir2 protein is a class III deacetylase which uses NAD as a cosubstrate. Mammalian Sir2 homologs have NAD-dependent histone deacetylase activity. Biochemical studies have shown that Sir2 can readily deacetylate the amino-terminals of histones H3 and H4 resulting in the formation of 1-O-acetyl-ADP-ribose and nicotinamide. It has recently been shown that additional copies of the C. elegans SIR2 homolog, sir2.1, and the D. melanogaster dSir2 gene greatly extend life span in those organisms. It is believed that Sir2 genes have evolved to enhance an organism's health and stress resistance to increase its chance of surviving adversity. SIRT3 is a homolog of SIRT1 that is conserved in prokaryotes and eukaryotes (P. Onyango et al. 2002). The SIRT3, SIRT4, and SIRT5 proteins are located within the mitochondrial matrix protein. It is known that SIRT3 is targeted to the mitochondrial cristae by a unique domain located at the N-terminus. SIRT3 has NAD+-dependent protein deacetylase activity and is ubiquitously expressed, particularly in metabolically active tissues. Upon transfer to the mitochondria, SIRT3 is believed to be cleaved into a smaller, active form by a mitochondrial matrix processing peptidase (MPP) (B. Schwer et al., 2002). Caloric restriction has been known for over 70 years to improve the health and extend the lifespan of mammals (Masoro, 2000). Yeast life span, like that of metazoans, is also extended by interventions that resemble caloric restriction, such as low glucose. Moreover, mutations that reduce the activity of the yeast glucose-responsive cAMP (adenosine 3′,5′-monophosphate)-dependent (PKA) pathway extend life span in wild type cells but not in mutant sir2 strains, demonstrating that SIR2 is likely to be a key downstream component of the caloric restriction pathway. Moreover, SIRT1 is implicated in a variety of disease states including diabetes, metabolic disorders (e.g., non-alcoholic fatty liver syndrome (NAFLS), non-alcoholic steatohepatitis (NASH)), and CNS disorders (multiple sclerosis (MS), amyotrophic lateral sclerosis (ALS), Parkinson's Disease (PD), Alzheimer's Disease (AD), and Huntington's Disease).

It has been shown that the isoprenoid compound resveratrol extends lifespan in yeast, worms, flies, and a short-lived species of fish. In rodents, resveratrol improves health, and prevents the early mortality associated with obesity. However, the precise mechanism of action has remained a subject of debate although it has generally been assumed to be through sirtuin activation. Dai et al. (2010) describes how Sirt1 may be activated by small molecules. Recently, Pacholec et al. (2010) have showed that resveratrol does not activate SIRT1 even against full-length protein substrates in the absence of a fluorophore. The sirtuin deacetylase activity requires the cofactor nicotinamide adenine dinucleotide (NAD, NAD+, or NADP, NADPH), and Hashimoto et al. (2010), Biogerontology Volume 11, Number 1, 31-43, have found that NAD extends nematode life suggesting that NAD has the capacity to extend lifespan regardless of species. NAD functions as a cofactor in over 200 redox reactions and as a substrate for three classes of enzymes: NAD-dependent deacetylases (Sirtuins), ADP-ribosyl transferases (most prominently, PARP-1), ADP cyclases (e.g. CD38), and GPR109a. NAD deficiency may result from excess histamine levels, and NAD deficiency disorders include alcoholism, drug addiction, violent behaviors, schizophrenia and multiple sclerosis, cf., e.g., Penberthy & Tsunoda, (Curr Pharm Des. 2009; 15(1): 64-99).

Thus, it is a purpose of the present invention to provide novel heterocyclic compounds which are useful as sirtuin modulators, activators or inhibitors. In addition, it is a purpose of the invention to provide novel compounds that may mimic at least one activity of nicotinamide adenine dinucleotide. Furthermore, it is a purpose of the present invention to provide novel pharmacologically active compounds for the treatment of diseases related to sirtuin and/or nicotinamide adenine dinucleotide deficiencies.

### SUMMARY OF THE INVENTION

Said purposes are fulfilled by the compounds of the present invention described by the general formula I

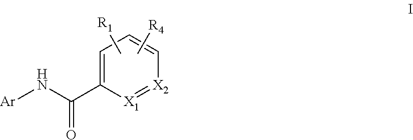

wherein

### X₁═N or C,

X₂═N or C, and X₁ # X₂, or one of X₁ and X₂ is absent resulting in a pyrrol ring,
R1 is a substituent selected from —C(═O)—R5, —C(═O)—N(CH₃)₂, —C(═O)—NHCH₃, —C(═O)—N(CH₃)₂,
and the aromatic ring system Ar may consist of a phenyl ring or a naphthyl ring having at least one substituent R2,
R2 is selected from the groups consisting of OH, NH₂ and SH;

In addition, Ar has an optional substituent R3 selected from the groups consisting of cyano, NO₂ and a halogen such as F, Cl, B, or R3 may be selected from the group consisting of —CONRxRy, —NHCOAlkyl, —COOH, —COOAlkyl,

—SOAlkyl, —SO₂Alkyl, and —NRxRyRz⁺; or from the group consisting of OH, —NRxRy, SH, Alkoxy, thioalkoxy, alkyl and aryl, wherein Rx, Ry and Rz represent an alkyl group, and the alkyl and alkoxy moieties are preferably of lower carbon chain lengths, such as C1-4, and the aryl moiety is preferably a phenyl;
R4 is an optional substituent selected from C1-4 lower alkyl such as methyl;
R5 is NH₂ optionally substituted with C1-4 lower alkyl such as CH₃, and a solvate, tautomer or isomer thereof including pharmaceutically acceptable salts, acid addition salts and base addition salts. The lower alkyl groups are preferably of straight carbon chains. In addition, heterocyclic compounds having an analogous chemical structure as the compounds of formula I, but wherein the central amide functionality is inverted, are also included in the present invention, Formula Ia:

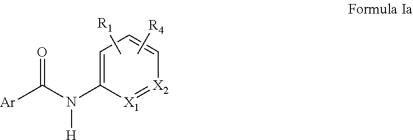

In these compounds the various substituents, including Ar, are as defined above. More particularly, the invention relates to compounds of Formula I and Ia, wherein Ar is a phenyl ring, R1 is —C(═O)—NH₂, R2 is OH in the 2 position, and R3=R4=H; or Ar is a naphthyl ring, R1 is —C(═O)—NH₂, R2 is OH preferably in the 2, 3, 6 or 8 position; and R3=R4=H, and solvates, tautomers or isomers thereof including pharmaceutically acceptable salts, acid addition salts and base addition salts.

### BRIEF DESCRIPTION OF THE DRAWINGS

Dose response studies were made using the assay where the initial concentration of each compound of the invention was reduced with three-fold aqueous dilutions, c.f. results in FIGS. 1 to 5 for the compounds 2, 9, 11, 13 and 17, respectively. The figures show percentage fluorescence as a function of Log C (log of compound concentration in μM). AC50 and IC50 values are calculated from the Log C value at 50% fluorescence.

FIG. 6 displays the kinetic solubility of compounds 3, 9, and 11 at 50 mM in phosphate buffer (pH=7.4).

FIG. 7 displays the stability of compounds 3, 9, and 11 in cryopreserved hepatocytes for 90 minutes.

FIG. 8 displays the protein binding of compounds 3, 9, and 11 in rat plasma and rat brain homogenate.

FIG. 9 demonstrates the efflux ratio of compounds 3, 9, and 11 in MDCK-MDR1 cells.

FIG. 10 displays the CYP inhibition of compounds 3, 9, 11, 19, 24 and 29 in human liver microsomes.

### DETAILED DESCRIPTION OF THE INVENTION

In one embodiment the compounds of the invention may further comprise a solubilizing group such as a phosphate group. In certain embodiments, compounds of the invention of formula I can be prepared as represented in the scheme below:

### General Synthetic Scheme a):

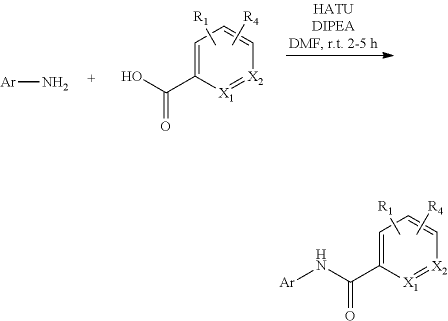

or, more specifically, in a more preferred embodiment, according to the general synthetic scheme b):

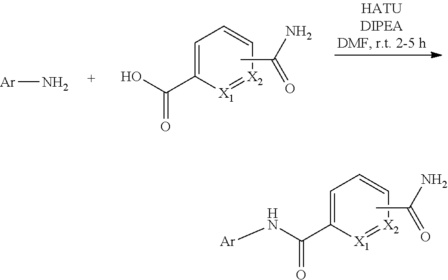

Wherein HATU is a coupling reagent that generates an azabenzotriazole ester (CAS Reg, No, 148893-10-1), DIPEA is diisopropylethylamine (a basic catalyst), DMF is dimethylformamide (a solvent), and r.t. is reaction time (generally 2 to 5 hours). The reaction may take place in solution phase, however, solid phase reactions are possible, such as is generally known by the skilled person in the art.

According to another embodiment, the present invention provides methods of producing the above-defined sirtuin-modulating compounds. The compounds may be synthesized using conventional techniques. Advantageously, these compounds are conveniently synthesized from readily available starting materials. Synthetic chemistry transformations and methodologies useful in synthesizing the compounds of formula I as well as of formulae II, III and IV described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

Preferred compounds of the invention include compounds selected from
2-N(2-hydroxyphenyl)pyridine-2,5-dicarboxamide,
5-N(2-hydroxyphenyl)pyridine-2,5-dicarboxamide,
2-N(6-hydroxy-1-naphthyl)pyridine-2,5-dicarboxamide,
5-N(5-hydroxy-1-naphthyl)pyridine-2,5-dicarboxamide,
5-N(6-hydroxy-1-naphthyl)pyridine-2,5-dicarboxamide,
5-N(4-hydroxy-1-naphthyl)pyridine-2,5-dicarboxamide, and a solvate, tautomer or isomer thereof including pharmaceutically acceptable salts and acid addition salts.

The invention further relates to a medical composition comprising an effective amount of a compound of formula I and a pharmaceutically acceptable carrier. Said medical composition may be pharmaceutically formulated according to a preferred route of administration, such as for intravenous, peroral, transdermal or subcutaneous, and intracranial administration and may be adapted for both topical and systemic administration. Thus, the composition may be in the liquid form, such as a solution, emulsion or suspension suitable for injection; or in the solid form, such as a powder or granulate, tablet, capsule, or sachet suitable for peroral administration or such as a suppository suitable for anal administration. For topical administration the composition may be in the form of, e.g. a self adhesive patch or an ointment or powder.

In addition, the invention relates to a method for treating a patient suffering from a sirtuin mediated disorder, such as medical disorders of brain, circadian rhythm and nervous system, anti-aging, diabetes, cancers, cardiovascular, fatty liver, inflammation, metabolic and obesity, but is not limited to such diseases, comprising administering to said patient an effective amount of a medical composition including a compound of formula I. Treatment regiments may include more than one daily administration.

### DEFINITIONS

As used herein, the following terms and phrases shall have the meanings set forth below. Unless defined otherwise, all technical and scientific terms used herein have the same meaning as commonly understood to one of ordinary skill in the art. The singular forms “a,” “an,” and “the” include plural reference unless the context clearly dictates otherwise.

The term “NAD” as used herein denotes the co-enzyme or cofactor nicotinamide adenine dinucleotide (variously abbreviated NAD, NAD+, including the phosphorylated forms NADP, NADP(H)). Apart as functioning as a co-factor in over 200 redox reactions NAD(P(H)) act as a substrate per se in various biochemical reactions. NAD functions as a cofactor in energy-producing catabolic reactions, such as the degradation of carbohydrates, fats, proteins, and alcohol, whereas NADP functions in anabolic reactions, such as the synthesis of cellular macromolecules including fatty acids and cholesterol [1]. As a co-factor NAD participates in oxidation-reduction (redox) reactions as hydride donor (NADH and NADPH) and acceptor (NAD (or NAD+) and NADP). Of all the NAD((P)H) specific molecular isoforms, it is specifically NAD and not NADH, NADP, nor NADPH that is the molecule most susceptible to deficiency under niacin-limiting conditions in bone marrow cells subjected to common oxidative stress. Nicotinic acid and nicotinamide (or niacin) are precursors for the biosynthesis of NAD. The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The term “bioavailable” when referring to a compound means that said compound, or a portion of the amount of compound administered, to be absorbed by, incorporated to, or otherwise physiologically available to a subject or patient to whom it is administered. “Biologically active portion of a sirtuin” refers to a portion of a sirtuin protein having a biological activity, such as the ability to deacetylate, including the ability to hydrolyze NAD+ into an ADP-ribose moiety and nicotinamide; ability to remove an acetyl group from a protein, which produces a deacetylated protein; and the ability to transfer the now available acetyl group to the ADP-RIBOSE MOIETY, which results in the formation of 2′-0-acetyl-ADP-ribose. (YANG et al., 2006, NAD metabolism and sirtuins: metabolic regulation of protein deacetylation in stress and toxicity. AAPS J 2006; 8:E632-E643). Because of this ability to deacetylate other proteins and hence activate a variety of important proteins, sirtuins play an important regulatory role in many biological processes. SIRT1 mediated deacetylase reactions are predominantly nuclear and target numerous proteins in a wide array of tissues, impacting the subsequent biological activity of these proteins. Biologically active portions of a sirtuin may comprise the core domain of sirtuins, cf. Hoff et al. (2006).

“Diabetes” refers herein to high blood sugar or ketoacidosis, as well as chronic, general metabolic abnormalities arising from an elevated blood glucose level or a decrease in glucose tolerance, i.a. Diabetes Mellitus Type I and II as well as various stages of insulin resistance.

The term “AC50” (half maximal effective activating concentration) is used herein as a measure of the potency of the compounds of the invention, and refers to the concentration of the compound which induces an enzymatic activation response halfway between the baseline and maximum following the required incubation time. Herein the SIRT1 activation response is measured as a FRET induced UV signal where maximal signal (100%) corresponds to no activation.

The term “IC50” (half maximal inhibitory concentration) is a measure of the effectiveness of a compound in inhibiting biological or biochemical function. This quantitative measure indicates how much of a particular drug or other substance (inhibitor) is needed to inhibit a given biological process (or component of a process, i.e. an enzyme, cell, cell receptor or microorganism) by half. It is commonly used as a measure of antagonist drug potency in pharmacological research.

The term “ED50” means the dose of a drug which produces 50% of its maximum response or effect, or alternatively, the dose which produces a pre-determined response in 50% of test subjects or preparations. The term “LD50” means the dose of a drug which is lethal in 50% of test subjects. The term “therapeutic index” is an art-recognized term which refers to the therapeutic index of a drug, defined as LD50/ED50. Sirtuin-modulating compounds that exhibit large therapeutic indexes are preferred. An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis. “Obese” individuals or individuals suffering from obesity are generally individuals having a body mass index (BMI) of at least 25 or greater. Obesity may be associated with insulin resistance.

The terms “parenteral administration” and “administered parenterally” refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion all of which may be useful in administration of the compounds of the invention. A “patient”, “subject”, “individual” or “host” may refer to either a human or a non-human animal.

The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be acceptable in the sense of being compatible with the subject composition and its components and not injurious to the patient. Some examples of materials which may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The terms “prophylactic” or “therapeutic” treatment are types of medical treatment referring to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom). “Sirtuin-activating compound” refers to a compound, such as a compound of Formulae I-IV as described herein, that increases at least one activity of a sirtuin protein, such as the deacetylation of a peptide substrate or, alternatively, said compound acts by directly or indirectly increasing the level of a sirtuin protein, e.g. through up-regulation of the corresponding gene transcription or inhibition of degradation pathways. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan of cells and organisms; increasing genomic stability; silencing transcription; and controlling the segregation of oxidized proteins between mother and daughter cells. “Sirtuin-inhibiting compound” refers to a compound that directly or indirectly decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. “Sirtuin-modulating compound” refers to a compound, such as a compound of Formulae I-IV as described herein which may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound. “Sirtuin protein” refers to a member of the sirtuin deacetylase protein family, or preferably to the sir2 family, which include yeast Sir2, C. elegans Sir-2.1, and human SIRT1, SIRT2 SIRT3, SIRT4, SIRT5, SIRT6 and SIRT7. Preferred sirtuins are those that share more similarities with SIRT1, such as human hSIRT1.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes. The term “therapeutic agent” refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human. The term “therapeutic effect” refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment. “Treating” a condition or disease refers to curing as well as ameliorating at least one symptom of the condition or disease. The term “vision impairment” refers to diminished vision, which is often only partially reversible or irreversible upon treatment (e.g., surgery). Particularly severe vision impairment is termed “blindness” or “vision loss”, which refers to a complete loss of vision, vision worse than 20/200 that cannot be improved with corrective lenses, or a visual field of less than 20 degrees diameter (10 degrees radius).

As used herein, the term “aryl” means a carbocyclic aromatic group, as for example phenyl, naphthyl, indenyl, indonyl, anthracenyl, flurorenyl, and the like.

The term “heterocyclic” refers to an aromatic cyclic group having one or more oxygen, nitrogen or sulfur atoms in the ring, as for example, furyl, thienyl, pyridyl, pyrrolyl, oxazolyl, thiazolyl, pyrazolyl, 1,2,3-triazolyl, indolyl, isoindolyl, quinolinyl, isoquinolinyl, and the like.

In one aspect, the invention provides novel sirtuin-modulating compounds for treating and/or preventing a wide variety of diseases and disorders including, for example, diseases or disorders related to aging or stress, diabetes, obesity, neurodegenerative diseases, ocular diseases and disorders, cardiovascular disease, blood clotting disorders, inflammation, cancer, and/or flushing, etc. Sirtuin-modulating compounds that increase the level and/or activity of a sirtuin protein (sirtuin activating compounds) are preferred herein and may additionally be used for treating a disease or disorder in a subject that would benefit from increased mitochondrial activity, for enhancing muscle performance, for increasing muscle ATP levels, or for treating or preventing muscle tissue damage associated with hypoxia or ischemia. Other compounds disclosed herein may be suitable for use in a pharmaceutical composition and/or one or more methods disclosed herein.

Sirtuin-modulating compounds of the invention advantageously modulate the level and/or activity of a sirtuin protein, particularly the deacetylase activity of the sirtuin protein with or without the necessity for the sirtuin to associate with its natural cofactor NAD/NAD+.

The compounds and salts described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan. The compounds and salts thereof can be present in amorphous or crystalline (including co-crystalline and polymorph) forms.

The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide). Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogen-phosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-I,4-dioate, hexyne-I,6-dioate, benzoate, chlorobenzoate, methylbenzoate, di nitrobenzoate, hydroxybenzoate, methoxy-benzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing the salts of this invention thus include sodium hydroxide, potassium hydroxide, ammonium hydroxide, potassium carbonate, and the like.

Sirtuin-modulating compounds described herein may also have one or more of the following characteristics: the compound may be essentially non-toxic to a cell or a subject. A sirtuin-modulating compound may promote deacetylation of the DNA repair factor Ku70; a sirtuin-modulating compound may promote deacetylation of RelA/p65 a subunit of the NF-κB transcription factor that regulates a wide range of cellular processes; a sirtuin modulating compound of the invention may sensitize cells to TNF-induced apoptosis, cf. Fan Yeung et al. (2004).

In certain embodiments, a sirtuin-modulating compound may have the ability to modulate one or more sirtuin protein homologs, such as, for example, one or more of human SIRT1, SIRT2, SIRT3, SIRT4, SIRT5, SIRT6, or SIRT7. In one embodiment, a sirtuin-modulating compound has the ability to modulate both a SIRT1 and another sirtuin protein such as SIRT3 protein. This may be desirable since SIRT3 is a known mitochondria-localized tumor suppressor required for maintenance of mitochondrial integrity and metabolism during stress. Thus, activation of both SIRT1 and SIRT3 may combine the benefits of SIRT1 deacetylation activity with a cancer fighting mechanism.

In certain embodiments, a sirtuin-modulating compound may have a binding affinity for a sirtuin protein of about 10⁻⁹M, 10⁻¹⁰M, 10⁻¹¹ M, 10⁻¹²M or less. A sirtuin-modulating compound of the invention may reduce (activator) or increase (inhibitor) the apparent rate constant Km of a sirtuin protein for its substrate or NAD+ cofactor by a factor of at least about 2, 3, 4, 5, 10, 20, 30, 50 or 100. In certain embodiments, Km values are determined using the assay described herein. A sirtuin-modulating compound may increase the Vmax of a sirtuin protein by a factor of at least about 2, 3, 4, 5, 10, 20, 30, 50 or 100. A sirtuin-modulating compound may have an ED50 for modulating the deacetylase activity of a SIRT1 and/or SIRT3 protein of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A sirtuin-modulating compound may modulate the deacetylase activity of a SIRT1 and/or SIRT3 protein by a factor of at least about 5, 10, 20, 30, 50, or 100, as measured in a cellular assay or in a cell based assay. A sirtuin-activating compound may cause at least about 10%, 30%, 50%, 80%, 2 fold, 5 fold, 10 fold, 50 fold or 100 fold greater induction of the deacetylase activity of a sirtuin protein relative to the same concentration of resveratrol. A sirtuin-modulating compound may have an ED50 for modulating SIRT5 that is at least about 10 fold, 20 fold, 30 fold, 50 fold greater than that for modulating SIRT1 and/or SIRT3.

### Exemplary Uses

In certain aspects, the invention provides methods for modulating the level and/or activity of a sirtuin protein and methods of use thereof. In certain embodiments, the invention provides methods for using sirtuin-modulating compounds wherein the sirtuin-modulating compounds activate a sirtuin protein, e.g., increase the level and/or activity of a sirtuin protein. Sirtuin-modulating compounds that increase the level and/or activity of a sirtuin protein may be useful for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. The methods comprise administering to a subject in need thereof a pharmaceutically effective amount of a sirtuin-modulating compound, e.g., a sirtuin-activating compound.

### Medical Uses

Sirtuins: (1) are proteins encoded by genes that control other genes, (2) respond in an epigenetic manner to a variety of environmental factors, and (3) are hypothesized to play a particular role in an organism's response to certain types of stress and toxicity. The SIRT1-7 genes have relevance for a number of human diseases:

### Anti-Aging/Lifespan Extension

The sirtuin system appears to be involved in mediating the increase in longevity produced by calorie restriction. Limited available evidence also connects increased expression of SIRT1 with increased lifespan and a more gradual aging process, as well as mitigation of symptoms of aging, in some species. As an example, mice that overexpress SIRT1 have an extended lifespan and maintain lower cholesterol, blood glucose, and insulin levels. They also showed increased numbers of mitochondria in their neurons.

Conversely, the lifespan of mice lacking SIRT1 is reduced under both normal and calorie-restricted conditions. Thus, In one embodiment, the invention provides a method of extending the lifespan of a cell or organism, extending the proliferative capacity of a cell or organism, slowing aging of a cell or organism, promoting the survival of a cell or organism, delaying cellular senescence in a cell or organism, mimicking the effects of calorie restriction, increasing the resistance of a cell or organism to stress, or preventing apoptosis of a cell, by contacting the cell with a sirtuin-modulating compound of the invention, such as a compound of formula I, that increases the level and/or activity of a sirtuin protein. In an exemplary embodiment, the methods comprise contacting the cell or organism with a sirtuin-activating compound. The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a sirtuin-modulating compound that increases the level and/or activity of a sirtuin protein to keep the cells, or progeny thereof, in culture for longer periods of time. Such cells can also be used for transplantation into a subject, e.g., after ex vivo modification. In one embodiment, cells that are intended to be preserved for long periods of time may be treated with a sirtuin-modulating compound that increases the level and/or activity of a sirtuin protein. The cells may be in suspension (e.g., blood cells, serum, biological growth media, etc.) or in tissues or organs. For example, blood collected from an individual for purposes of transfusion may be treated with a sirtuin-modulating compound that increases the level and/or activity of a sirtuin protein to preserve the blood cells for longer periods of time. Additionally, blood to be used for forensic purposes may also be preserved using a sirtuin-modulating compound that increases the level and/or activity of a sirtuin protein.

In another embodiment, the sirtuin-modulating compounds of the invention that increase the level and/or activity of a sirtuin protein may be useful as a reagent for treatment of cells and tissues, e.g. in a pretreatment of organs, tissues and cells useful for transplantation or cell therapy, including, for example, solid tissue grafts, organ transplants, cell suspensions, stem cells, bone marrow cells, etc. The cells or tissue may be treated with the sirtuin-modulating compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a sirtuin-modulating compound or may have a subset of cells/tissue treated locally with a sirtuin-modulating compound that increases the level and/or activity of a sirtuin protein.

In yet other embodiments, cells or organisms may be treated with a sirtuin-modulating compound that increases the level and/or activity of a sirtuin protein in vivo, e.g., to increase their lifespan or prevent apoptosis. For example, skin can be protected from aging (e.g., developing wrinkles, loss of elasticity, etc.) by treating skin or epithelial cells with a sirtuin-modulating compound that increases the level and/or activity of a sirtuin protein. In an exemplary embodiment, skin is contacted with a pharmaceutical or cosmetic composition comprising a sirtuin-modulating compound that increases the level and/or activity of a sirtuin protein. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, sirtuin-modulating compounds that increase the level and/or activity of a sirtuin protein may be used for the treatment of wounds and/or burns to promote healing, including, for example, first-, second- or third-degree burns and/or a thermal, chemical or electrical burns. The formulations may be administered topically, to the skin or mucosal tissue. Topical formulations comprising one or more sirtuin-modulating compounds that increase the level and/or activity of a sirtuin protein may also be used as preventive, e.g., chemopreventive, compositions. When used in a chemopreventive method, susceptible skin is treated prior to any visible condition in a particular individual. Sirtuin-modulating compounds may be delivered locally or systemically to a subject. In one embodiment, a sirtuin-modulating compound is delivered locally to a tissue or organ of a subject by injection, topical formulation, etc.

In another embodiment, a sirtuin-modulating compound that increases the level and/or activity of a sirtuin protein may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject.

In yet another embodiment, a sirtuin-modulating compound that increases the level and/or activity of a sirtuin protein may be administered to a subject in order to generally increase the lifespan of its cells and to protect its cells against stress and/or against apoptosis.

Sirtuin-modulating compounds that increase the level and/or activity of a sirtuin protein may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. Sirtuin-modulating compounds that increase the level and/or activity of a sirtuin protein can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

Sirtuin-modulating compounds that increase the level and/or activity of a sirtuin protein can also be administered to a subject suffering from an acute disease, e.g., damage to an organ or tissue, e.g., a subject suffering from stroke or myocardial infarction or a subject suffering from a spinal cord injury. Sirtuin-modulating compounds that increase the level and/or activity of a sirtuin protein may also be used to repair an alcoholic's liver.

### Obesity and Metabolic Syndrome

Sirtuins are thought to play a role in obesity and obesity-related issues. Evidence for this role comes from emerging understanding of the regulatory role sirtuins play in metabolic pathways and adaptations linked with obesity and aspects of metabolic syndrome. These include the expression of adipocyte cytokines (adipokines), the maturation of fat cells, insulin secretion and tissue sensitivity, modulation of plasma glucose levels, cholesterol and lipid homeostasis, and mitochondrial energy capacity.

### SIRT1

SIRT1, for example, is involved in the regulating the expression of adipokines such as adiponectin and tumor necrosis factor, has been linked to hypothalamic control of energy balance, plays a role in adipogenesis, and is involved in the regulation of lipolysis and fatty acid mobilization in response to fasting. Evidence from animal experiments where sirtuins are over- or underexpressed, and from limited human evidence, also suggests a role for sirtuins in obesity. SIRT1 is highly expressed in the hypothalamus, where it appears to be involved in regulating energy homeostasis, food intake, and body weight. Fasting upregulates hypothalamic SIRT1 expression, which is associated with the fasting-induced increase in hunger, and is presumably part of the complex adaptations against calorie restriction-induced weight loss. Conversely, pharmacological inhibition of hypothalamic SIRT1 decreases food intake and body weight gain in rodents, suggesting that the hypothalamic SIRT1 inhibition might suppress appetite. In mice, calorie restriction induces a complex pattern of physiological and behavioral adaptations, including an increase in activity and food seeking; SIRT1 is required for these behavioral adaptations. In mice, decreased SIRT1 expression in adipose tissue is associated with obesity. In both db/db mice (leptin resistant mice) and mice that have become obese from eating a high-fat diet, SIRT1 expression in adipose tissue is low. Circumstances that result in SIRT1 underexpression in white adipose enhance adipogenesis and, under fasting conditions, compromise mobilization of fatty acids from white adipocytes. Conversely, circumstances that promote white adipose SIRT1 overexpression are characterized by attenuated adipogenesis and increased lipolysis. Experiments with transgenic mice that were bred to moderately overexpress SIRT1 in several tissues also suggest a role for SIRT1 in protecting against obesity. Transgenic mice with greater SIRT1 expression are leaner than littermate controls and have reduced levels of cholesterol, adipokines, insulin and fasting glucose. Reduced adiposity of these transgenic mice appears to be due to systemic weight regulation that results in decreased whole-body energy requirements, evidenced by the decreased food intake observed in these animals. Although another study did not observe an anti-obesity effect of SIRT1 overexpression in transgenic mice fed a high-fat diet, these mice were protected against some metabolic effects of this diet. Benefits of SIRT1 overexpression included less inflammation, better glucose tolerance, and almost complete protection against hepatic steatosis. SIRT1 expression has strong links to insulin sensitivity. Reports indicate that SIRT1 is down-regulated in highly insulin resistant cells, while inducing its expression in these cells increases insulin sensitivity. In skeletal muscle, SIRT1 contributes to the improvement of insulin sensitivity through the transcriptional repression of the protein tyrosine phosphatase 1B (PTP1B) gene. In adipocytes, SIRT1 regulates insulin-stimulated glucose uptake and GLUT4 translocation, with greater SIRT1 activity attenuating insulin resistance. In various rodents models of insulin resistance and diabetes, SIRT1 transgenic mice display improved glucose tolerance and insulin sensitivity, due in part to decreased hepatic glucose production and increased hepatic insulin sensitivity. SIRT1 expression appears to improve pancreatic beta-cell function. In beta-cell lines in which SIRT1 expression is inhibited, insulin secretion is blunted. Conversely, increased expression of SIRT1 promotes improved insulin secretion, cf. also Yoshizaki et al. 2010. These in vitro responses mirror what has been observed in vivo. In transgenic mice, bred to overexpress SIRT1 in pancreatic beta-cells, there is enhanced glucose-stimulated insulin secretion and improved glucose tolerance. This improvement of beta-cell function persists through the aging process and when these mice are fed high-fat diets. SIRT1 also regulates cholesterol metabolism by deacetylating and activating LXRalpha, a nuclear receptor involved in cholesterol and lipid homeostasis, cf. also Walker et al. 2010.

### Other Sirtuins

Less research has been conducted on the other members of the sirtuin family in conditions associated with obesity. The limited evidence suggests that SIRT2 is the most abundant sirtuin in adipocytes, where it appears to be involved in adipogenesis-adipocyte formation. Overexpression of SIRT2 inhibits preadipocyte differentiation into adipocytes, while decreased SIRT2 expression promotes adipogenesis. SIRT3 appears to influence both ATP formation (fatty acid oxidation) and adaptive thermogenesis. In mice lacking SIRT3, fatty acid oxidation disorders emerge during fasting, including reduced ATP levels. These mice also demonstrate a generalized intolerance to cold exposure during fasting, suggesting a disordered thermogenic response from brown adipose tissue. SIRT4 is expressed in beta-cells in the islets of Langerhans and is thought to play a role in mitochondrial regulation of insulin secretion. SIRT6 influences the expression of a variety of glycolytic genes, including genes involved in glucose uptake, glycolysis, and mitochondrial respiration. It appears to be a critical element of glucose homeostasis, with SIRT6-deficient mice developing a lethal hypoglycemia early in life. SIRT6 might also play a role in the mouse response to a high-fat diet. Transgenic mice bred to overexpress SIRT6 accumulate significantly less visceral fat and have much lower LDL-cholesterol and triglyceride levels when fed a high-fat diet compared to controls. They also display enhanced glucose tolerance and improved glucose-stimulated insulin secretion.

### Evidence in Humans

In humans, available information on sirtuin interaction with weight has come from observational or calorie restriction studies. In a study of SIRT1 mRNA expression in lean and obese women, lean women were reported to have more than two-fold higher SIRT1 expression in subcutaneous adipose tissue compared to obese women. In another study by Rutanen et al (2010), adipose tissue SIRT1 mRNA expression had a positive association with energy expenditure and insulin sensitivity in 247 non-diabetic offspring of type 2 diabetic patients. In a third study, SIRT1-SIRT7 gene and protein expression were determined in peripheral blood mononuclear cells from 54 subjects (41 with normal glucose tolerance and 13 with metabolic syndrome). Insulin resistance and metabolic syndrome were associated with low SIRT1 protein expression. In these studies, SIRT1 expression has a negative association with obesity or issues related to obesity; however, whether increased SIRT1 is involved in protecting against obesity, is a marker for obesity resistance, or is altered in response to ongoing dietary, lifestyle, or environmental factors, has not been established and cannot be determined from the existing evidence. What human evidence does make clear is that, similar to other species including other mammals, human sirtuin expression is sensitive to changes in calorie intake. SIRT1 mRNA was measured in adipose tissue biopsies from nine human volunteers before and after six days of total fasting. Levels in subcutaneous adipose tissue increased more than two-fold with fasting. In another study, muscle biopsies were obtained at baseline and on day 21 from 11 non-obese men and women who underwent three weeks of alternate day fasting; a statistically significant increase in muscle SIRT1 mRNA expression was observed. In a third study, diet-induced changes in adipose tissue gene expression were assessed in two sets of 47 obese women who were placed on either a low-fat (high-carbohydrate) or a moderate-fat (low-carbohydrate) hypoenergetic diet for 10 weeks. One thousand genes, including sirtuin genes, were regulated by energy restriction. SIRT3 gene expression appeared to be sensitive to the fat-to-carbohydrate ratio of a restricted calorie diet, with increased expression during moderate-fat diet.

### Weight Control

Thus, the compounds of the invention being, sirtuin-modulating compounds that increase the level and/or activity of a sirtuin protein may be used for treating or preventing weight gain or obesity in a subject. For example, sirtuin-modulating compounds that increase the level and/or activity of a sirtuin protein may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof. In yet other embodiments, sirtuin-modulating compounds that increase the level and/or activity of a sirtuin protein may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Finally, patients with AIDS can develop lipodystrophy or insulin resistance in response to combination therapies for AIDS.

In another embodiment, sirtuin-modulating compounds that increase the level and/or activity of a sirtuin protein may be used for inhibiting adipogenesis or fat cell differentiation, whether in vitro or in vivo. Such methods may be used for treating or preventing obesity.

In other embodiments, sirtuin-modulating compounds that increase the level and/or activity of a sirtuin protein may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.” In an exemplary embodiment, sirtuin-modulating compounds that increase the level and/or activity of a sirtuin protein may be administered as a combination therapy for treating or preventing weight gain or obesity. For example, one or more sirtuin-modulating compounds that increase the level and/or activity of a sirtuin protein may be administered in combination with one or more anti-obesity agents.

In another embodiment, sirtuin-modulating compounds that increase the level and/or activity of a sirtuin protein may be administered to reduce drug-induced weight gain. For example, a sirtuin-modulating compound that increases the level and/or activity of a sirtuin protein may be administered as a combination therapy with medications that may stimulate appetite or cause weight gain, in particular, weight gain due to factors other than water retention.

### Metabolic Disorders

In another aspect, sirtuin-modulating compounds that increase the level and/or activity of a sirtuin protein may be used for treating or preventing a metabolic disorder, such as insulin-resistance, a pre-diabetic state, type II diabetes, and/or complications thereof. Administration of a sirtuin-modulating compounds that increases the level and/or activity of a sirtuin protein may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, sirtuin-modulating compounds that increase the level and/or activity of a sirtuin protein may be administered as a combination therapy for treating or preventing a metabolic disorder. For example, one or more sirtuin-modulating compounds that increase the level and/or activity of a sirtuin protein may be administered in combination with one or more anti-diabetic agents.

### Fatty Liver Disease

The sirtuin system has a variety of links to alcoholic and nonalcoholic hepatic steatosis. SIRT1 In general, SIRT1 expression has a negative association with fatty infiltration of the liver in both rodents and humans. In rodents, these associations exist for nonalcoholic and alcoholic hepatic steatosis and appear to be related to inflammation and sirtuin interactions with liver fatty acid oxidation and transport. Sirtuin-steatosis interactions appear to be mediated, at least in part, by sirtuin deacetylation of other proteins, which subsequently modulates the activity of these proteins and their metabolic targets. For example, in a cell model of hepatic fatty infiltration, SIRT1 protect against hepatic fat deposition via induction of FOXO1 expression and repression of SREBP1 expression. It has also been proposed that sirtuin effects on the PPARalpha/PGC-1alpha signaling axis might be involved in the protective association. In rodents, a high-fat diet plays a significant role in interactions with SIRT1 and nonalcoholic hepatic steatosis. Reduced expression of hepatic SIRT1 proteins appears to predispose mice to high-fat diet induced hepatic steatosis, while increased expression appears to protect against steatosis; this has been demonstrated in several studies. When mice, bred to have reduced expression of hepatic SIRT1, were fed a low-fat diet (5% fat), they were no more likely to have manifestations of liver disease than normal mice. However, as dietary fat levels were increased in the mice with reduced hepatic SIRT1 expression, there was a corresponding increase in hepatic steatosis, with higher levels of dietary fat intake causing worse steatosis. These mice, in addition to significant increase in hepatic steatosis, experienced increased liver inflammation and hepatic lipogenesis, with a reduction in fat transport. As mentioned previously, sirtuins are both a regulating and a regulated protein. Deleted in breast cancer-1 (DBC1) is one protein with an established ability to regulate SIRT1. Mice bred to have a genetic deletion of DBC1 express increased SIRT1 activity in several tissues, including the liver. When these mice are fed a high-fat diet, they become obese but do not develop the hepatic steatosis and inflammation typically caused by this diet and that generally accompanies diet-induced obesity. While increased SIRT1 expression appears to have a protective role against diet-induced hepatic steatosis, evidence also suggests that a high-fat diet can reduce SIRT1 expression. This suggests that an inability to counter the high-fat diet-induced downregulation of SIRT1 might play a role in susceptibility to diet-induced hepatic steatosis.

### Other Sirtuins

Evidence of interactions with the other members of the sirtuin family and the fatty liver is sparse. In vitro, the number of lipid droplets in human hepatic cells overexpressing SIRT3 was significantly lower than in control cells. Decreasing SIRT3 expression promoted lipid accumulation in these cells. Under in vivo fasting conditions, SIRT3 expression prevents the accumulation of lipid droplets in hepatis cells. Chronic alcohol-feeding also reduced SIRT5.

### Evidence in Humans

In humans, SIRT1 overexpression in visceral adipose tissue was associated with severity of hepatic steatosis. In this study, morbidly obese individuals were divided into two groups—one with moderate hepatic steatosis and the other with severe steatosis. When comparing the two groups, a decrease of SIRT1 mRNA in visceral adipose tissue was detected in samples taken from the group with severe hepatic steatosis. Statistical analysis also revealed a positive correlation between mRNA expression of SIRT1 and homeostasis model assessment for insulin resistance (HOMA-IR). The researchers did not explore whether the downregulation of SIRT1 mRNA expression in visceral adipose tissue was promoting steatosis in these obese individuals or a response to severe steatosis.

### Cardiovascular System

In vitro and in vivo evidence suggests a role for several of the sirtuins in the cardiovascular system. SIRT1 appears to play a regulatory role in endothelial function. It is highly expressed in vasculature, especially during periods of active blood vessel growth and vascular remodeling, when it appears to be involved in angiogenic activity of endothelial cells. SIRT1 promotes endothelium dependent vasodilatation and regenerative functions in endothelial and smooth muscle cells of the vascular wall by targeting endothelial nitric oxide synthase for deacetylation, which stimulates the activity of this enzyme and increases endothelial nitric oxide production. If SIRT1 deacetylation is inhibited in endothelial tissue, nitric oxide synthase acetylation predominates, nitric oxide production decreases, and vasodilatation is impaired. SIRT1 might also play a significant role on endothelial function when blood glucose is elevated. Treatment of human endothelial cells with glucose decreases SIRT1 expression, induces endothelial dysfunction, and accelerates endothelial senescence. Increasing SIRT1 activity inhibits this glucose-induced endothelial senescence and dysfunction. These effects were also seen in vivo; activation of SIRT1 prevented hyperglycemia-induced vascular cell senescence and protected against vascular dysfunction in mice. SIRT1 might play a role in countering atherosclerosis due to its reported regulation of tissue metalloproteinase 3 (TIMP3). TIMP3 is an endogenous enzyme that counters vascular inflammation and is involved in the prevention of atherosclerosis. SIRT1 activity is also reportedly decreased in atherosclerotic plaques of subjects with type 2 diabetes—a decrease associated with reduced TIMP3 expression. SIRT1, SIRT3, and SIRT7 are expressed in cardiomyocytes, are upregulated during stress conditions (presumably as an adaptation to counter the stress), and appear to play a critical role in promoting cardiomyocyte resistance to stress and toxicity. Cardiomyocyte protection appear to occur because of sirtuin deacetylation of other proteins, with the relative balance between acetylation and deacetylation of these targeted proteins influencing whether cardiomyocytes survive under stressful conditions. Sitruins also protect cardiomyocytes by activating antioxidant-encoding genes (including manganese superoxide dismutase and catalase) that decrease cellular levels of reactive oxygen species. Circumstances that result in decreased SIRT1 are associated with reduced cardiac function. For example, in mice with chronic type 1 diabetes, the enzymatic activity of cardiac SIRT1 is reduced, which contributes to reduced cardiac function and diabetic cardiomyopathy. While increased cardiomyocyte SIRT1 expression and activity appear to be an adaptation to stress and toxicity, limited evidence suggest that extremes of increased expression might not be desirable. Transgenic mice bred to have 2.5- to 7.5-fold hear-specific SIRT1 overexpression were protected against oxidative stress. Age-dependent increases in cardiac hypertrophy, apoptosis/fibrosis, cardiac dysfunction, and expression of senescence markers were consequently attenuated. However, a 12.5-fold overexpression of heart-specific SIRT1 increased oxidative stress, apoptosis, and hypertrophy, and decreased cardiac functions, stimulating the development of cardiomyopathy. In this case, rather than being protective and conferring resistance to age-related problems, the highest levels of SIRT1 expression promoted pathology. This may be a result of higher SIRT1 consumption of cellular NAD+ exceeding the supply or unbalanced acetylation/deacetylation activities. Whatever the mechanism, these results suggest that the cardioprotective effects of heart-specific SIRT1 expression may be biphasic, with too much expression resulting in diminishing returns.

Thus, the invention provides a method for treating and/or preventing a cardiovascular disease by administering to a subject in need thereof a sirtuin-modulating compound that increases the level and/or activity of a sirtuin protein. Cardiovascular diseases that can be treated or prevented using the sirtuin-modulating compounds that increase the level and/or activity of a sirtuin protein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The sirtuin-modulating compounds that increase the level and/or activity of a sirtuin protein may also be used for increasing HDL levels in plasma of an individual. Yet other disorders that may be treated with sirtuin-modulating compounds that increase the level and/or activity of a sirtuin protein include restenosis, e.g., following coronary intervention, and disorders relating to an abnormal level of high density and low density cholesterol.

In one embodiment, a sirtuin-modulating compound that increases the level and/or activity of a sirtuin protein may be administered as part of a combination therapeutic with another cardiovascular agent, e.g. an anti-arrhythmia agent.

### Other Sirtuins

The importance of other sirtuins for cardiac function is apparent in SIRT3-deficient mice. In these mice, basal levels of ATP in the heart, kidney, and liver are reduced by more than 50 percent, and mitochondrial protein acetylation is markedly elevated in these same tissues. These mice also show signs of cardiac hypertrophy and interstitial fibrosis at age eight weeks and develop severe cardiac hypertrophy in response to hypertrophic stimuli. Conversely, transgenic mice that overexpress SIRT3 are protected from stimuli-induced cardiac hypertrophy. SIRT7 also appears to be critical for cardiac function. SIRT7-deficient mice have reduced mean and maximum life spans. Their hearts are characterized by extensive fibrosis, diminished resistance to oxidative and genotoxic stress, and a high basal rate of apoptosis resulting in cardiac hypertrophy and inflammatory cardiomyopathy.

### Brain and Nervous System

Several sirtuins expressed in the mammalian brain appear to play very different roles and respond in dissimilar ways to stress and toxicity. For example Pfister J A, Ma C, Morrison B E, D'Mello S R (2008) reported that SIRT1 protects neurons against apoptosis, while SIRT2, SIRT3 and SIRT6 induce apoptosis in otherwise healthy neurons. SIRT5 has a dual role. In neurons, where it is located in both the nucleus and cytoplasm, it exerts a protective effect; however, in a subset of neurons where it is located in the mitochondria, it promotes neuronal death. While all these sirtuins appear to impact neurons, almost all research has focused on SIRT1 and SIRT2. SIRT1 is ubiquitously present in areas of the brain that are especially susceptible to age-related neurodegenerative states (e.g. the prefrontal cortex, hippocampus, and basal ganglia). SIRT1 is also broadly distributed in neurons that are most susceptible to senescence injury. Calorie restriction results in up-regulation of SIRT1 in some regions of the brain (such as the hypothalamus) and down-regulation in others. In mice undergoing calorie restriction, there is an attenuation of beta-amyloid content in the aging brain. This effect can be reproduced in mouse neurons in vitro by manipulating cellular SIRT1 expression/activity, suggesting that it is a SIRT1-dependent process and that SIRT1 up-regulation might be protective under some types of nutritional stress. SIRT1 is up-regulated in primary neurons challenged with some types of neurotoxic insults. However, in transgenic mice created to overexpress human SIRT1 in neurons, the neuronal overexpression of SIRT1 had no neuroprotective effects against damage induced by ischemia or 1-methyl-4-phenyl-1,2,3,6-tetrahydropyridine. Evidence suggests that SIRT1 is up-regulated in the brain in mouse models of Alzheimer's disease (AD) and amyotrophic lateral sclerosis (ALS). In cell-based models of these conditions, increased SIRT1 promotes neuronal survival. In animal models of AD, cortical SIRT1 reduction parallels the accumulation of tau. In humans with AD, SIRT1 levels also reportedly decreased in the parietal cortex but not in the cerebellum. Lower cortical SIRT1 was correlated with the duration of symptoms, lower global cognition scores, and accumulation of amyloid-beta and tau in the cerebral cortex. SIRT2, the most predominantly expressed sirtuin in the human brain, is enriched in brain oligodendrocytes, where it is thought to be involved in differentiation, maturation, and remodeling. SIRT2 is also highly expressed in post-mitotic neurons and glial cells. In the brain and other tissues, SIRT2 acts as a tubulin deacetylase, which inhibits growth in postmitotic neurons and helps protect neuronal cells against mitotic stress. SIRT2 is also highly expressed in the myelin sheath, where alpha-tubulin is its main protein target. Decreasing expression of SIRT2 in myelin increases alpha-tubulin acetylation and myelin basic protein expression; increasing expression of SIRT2 has the opposite effect. Under some experimental circumstances SIRT2 inhibition appears to be neuroprotective. Inhibition of SIRT2 activity also protects against dopaminergic cell death in vitro and in Drosophila model of Parkinson's disease. Under other circumstances it might be advantageous to express SIRT2. For example, SIRT2 is reportedly reduced in some human brain tumor cell lines, which apparently causes a relative loss of tumor suppressor activity via its role in protein deacetylation.

Thus, In certain aspects, sirtuin-modulating compounds of the invention that increase the level and/or activity of a sirtuin protein can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a CNS disorder that results in memory loss, unusual behavior, personality changes, and a decline in thinking abilities. These losses are related to the death of specific types of brain cells and the breakdown of connections and their supporting network (e.g. glial cells) between them. The earliest symptoms include loss of recent memory, faulty judgment, and changes in personality. PD is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. HD is another neurodegenerative disease that causes uncontrolled movements, loss of intellectual faculties, and emotional disturbance. Tay-Sachs disease and Sandhoff disease are glycolipid storage diseases where GM2 ganglioside and related glycolipidssubstrates for β-hexosaminidase accumulate in the nervous system and trigger acute neurodegeneration. It is well-known that apoptosis plays a role in AIDS pathogenesis in the immune system. However, HIV-I also induces neurological disease, which can be treated with sirtuin-modulating compounds of the invention. Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. Sirtuin-modulating compounds that increase the level and/or activity of a sirtuin protein may be useful for treating or preventing neuronal loss due to these prior diseases.

In another embodiment, a sirtuin-modulating compound that increases the level and/or activity of a sirtuin protein may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. Those with distal axonopathies usually present with symmetrical glove-stocking sensori-motor disturbances. Deep tendon reflexes and autonomic nervous system (ANS) functions are also lost or diminished in affected areas. Diabetic neuropathies are relatively common conditions which may include third nerve palsy; mononeuropathy; mononeuritis multiplex; diabetic amyotrophy; a painful polyneuropathy; autonomic neuropathy; and thoracoabdominal neuropathy. Peripheral neuropathy is the medical term for damage to nerves of the peripheral nervous system, which may be caused either by diseases of the nerve or from the side-effects of systemic illness. Major causes of peripheral neuropathy include seizures, nutritional deficiencies, and HIV, though diabetes is the most likely cause.

In an exemplary embodiment, a sirtuin-modulating compound that increases the level and/or activity of a sirtuin protein may be used to treat or prevent multiple sclerosis (MS), including relapsing MS and monosymptomatic MS, and other demyelinating conditions, such as, for example, chronic inflammatory demyelinating polyneuropathy (CIDP), or symptoms associated therewith.

In yet another embodiment, a sirtuin-modulating compound that increases the level and/or activity of a sirtuin protein may be used to treat trauma to the nerves, including, trauma due to disease, injury (including surgical intervention), or environmental trauma (e.g., neurotoxins, alcoholism, etc.).

Sirtuin-modulating compounds that increase the level and/or activity of a sirtuin protein may also be useful to prevent, treat, and alleviate symptoms of various PNS disorders. The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used. PNS diseases treatable with sirtuin-modulating compounds that increase the level and/or activity of a sirtuin protein include: diabetes, leprosy, Charcot-Marie-Tooth disease, Guillain-Barre syndrome and Brachial Plexus Neuropathies (diseases of the cervical and first thoracic roots, nerve trunks, cords, and peripheral nerve components of the brachial plexus. In another embodiment, a sirtuin activating compound may be used to treat or prevent a polyglutamine disease. Exemplary polyglutamine diseases include spinobulbar muscular atrophy (Kennedy disease), Huntington's Disease (HD), dentatorubral-pallidoluysian atrophy (Haw River syndrome), spinocerebellar ataxia types 1, 2, 3, 6, 7 and 17.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell. Another aspect encompasses administrating a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. In another alternative of this embodiment, the stroke is a cerebellar stroke. In still another embodiment, the stroke is an embolic stroke. In yet another alternative, the stroke may be a hemorrhagic stroke. In a further embodiment, the stroke is a thrombotic stroke.

In yet another aspect, a sirtuin activating compound may be administered to reduce infarct size of the ischemic core following a central nervous system ischemic condition. Moreover, a sirtuin activating compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents.

### Cancer

Some evidence suggests SIRT1 is a tumor promoter, including increased SIRT1 expression in some cancers, and its role in deacetylating (and hence presumably deactivating) proteins like p53, p300, and foxhead transcription factors that are involved in tumor suppression and DNA repair. Conversely, other cancers have decreased expression of SIRT1. Other indications of SIRT1 as a tumor suppressor come from experimental results of mouse/cancer models in which SIRT1 is intentionally under-(tumorigenesis increases) or overexpressed (tumorigenesis attenuated). SIRT1 also exerts a positive influence on other proteins and processes that result in suppression of tumor growth and enhanced DNA repair. SIRT3 also appears to have both tumor promotion and tumor suppression effects. Although it is capable of deacetylating p53, it is involved in supporting pro-apoptotic processes by targeting other proteins for deacetylation and functions as a tumor suppressor by enhancing the expression of mitochondrial antioxidant enzymes. Mice lacking SIRT3 express genomic instability and develop tumors. Conflicting evidence exists, even within the same cancer tissue type. An association between increased SIRT3 and node-positive breast cancer has been reported (Ashraf et al. British Journal of Cancer (2006) 95, 1056-1061. doi:10.1038/sj.bjc.6603384, Published online 26 Sep. 2006), while Kim et al. (Volume 17, Issue 1, 19 Jan. 2010, Pages 41-52) reported reduced SIRT3 levels in breast (and other cancers) and noted that mice lacking SIRT3 develop mammary tumors.

Thus, Sirtuin-modulating compounds may also be used for treating and/or preventing cancer. In certain embodiments, sirtuin-modulating compounds that increase the level and/or activity of a sirtuin protein may be used for treating and/or preventing cancer. Calorie restriction has been linked to a reduction in the incidence of age-related disorders including cancer. Accordingly, an increase in the level and/or activity of a sirtuin protein may be useful for treating and/or preventing the incidence of age-related disorders, such as, for example, cancer. Exemplary cancers that may be treated using a sirtuin-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Li et al. (Cancer Cell, Volume 21, Issue 2, 266-281 (2012)) showed that activation of p53 via SIRT1 inhibition represents a potential approach to target CML LSC. BCR-ABL tyrosine kinase inhibitors (TKI) fail to eliminate quiescent leukemia stem cells (LSC) in chronic myelogenous leukemia (CML). Thus, strategies targeting LSC are required to achieve cure. The study showed that the NAD+-dependent deacetylase SIRT1 is overexpressed in human CML LSC. Pharmacological inhibition of SIRT1 or SIRT1 knockdown increased apoptosis in LSC of chronic phase and blast crisis CML and reduced their growth in vitro and in vivo. SIRT1 effects were enhanced in combination with the BCR-ABL TKI imatinib. SIRT1 inhibition increased p53 acetylation and transcriptional activity in CML progenitors, and the inhibitory effects of SIRT1 targeting on CML cells depended on p53 expression and acetylation.

Benign cell growth, e.g., warts, can also be treated. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-I associated malignant and benign disorders can also be treated by administration of sirtuin-modulating compound. Alternatively, cells can be obtained from a subject, treated ex vivo to remove certain undesirable cells, e.g., cancer cells, and administered back to the same or a different subject. Chemotherapeutic agents may be co-administered with modulating compounds described herein as having anti-cancer activity, e.g., compounds that induce apoptosis, compounds that reduce lifespan or compounds that render cells sensitive to stress. Chemotherapeutic agents may be used by themselves with a sirtuin-modulating compound described herein as inducing cell death or reducing lifespan or increasing sensitivity to stress and/or in combination with other chemotherapeutics agents. In addition to conventional chemotherapeutics, the sirtuin-modulating compounds described herein may also be used with antisense RNA, RNAi or other polynucleotides to inhibit the expression of the cellular components that contribute to unwanted cellular proliferation. Combination therapies comprising sirtuin-modulating compounds and a conventional chemotherapeutic agent may be advantageous over combination therapies known in the art because the combination allows the conventional chemotherapeutic agent to exert greater effect at lower dosage. In a preferred embodiment, the effective dose (ED50) for a chemotherapeutic agent, or combination of conventional chemotherapeutic agents, when used in combination with a sirtuin-modulating compound is at least 2 fold less than the ED50 for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a sirtuin-modulating compound described herein can be at least 2 fold greater than the TI for conventional chemotherapeutic regimen alone, and even more preferably at 5 fold, 10 fold or even 25 fold greater. Sirtuin-modulating compounds the invention that increase the level and/or activity of a sirtuin protein may be administered to subjects who have recently received or are likely to receive a dose of radiation or toxin. In one embodiment, the dose of radiation or toxin is received as part of a work-related or medical procedure, e.g., administered as a prophylactic measure. In another embodiment, the radiation or toxin exposure is received unintentionally. In such a case, the compound is preferably administered as soon as possible after the exposure to inhibit apoptosis and the subsequent development of acute radiation syndrome.

Other Sirtuins Although less is known about the other sirtuins and cancer, several have functions that suggest a role in cancer prevention. SIRT5 appears to regulate DNA repair and influences apoptosis. SIRT6 is involved in regulating chromatin structure, maintaining telomere integrity and genomic stability, and repairing DNA. SIRT7 promotes ribosomal gene (rDNA) transcription factors and has anti-proliferative effects.

### Protective Response to Certain Forms of Stress and Toxicity

Sirtuin expression is thought to be a protective response to certain forms of stress and toxicity. Some cancer therapies, including radiation and certain forms of chemotherapy, are genotoxic. Limited experimental evidence suggests that the sirtuin system might respond to these treatments to protect cells against them, which might also potentially interfere with the clinical efficacy of these treatments. For example, exposure of cells to radiation caused an increase in SIRT1 and a corresponding increase in DNA repair. Experimentally-induced overexpression of SIRT1 resulted in a greater increase in repair of DNA strand breakages produced by the radiation. Conversely, inhibiting SIRT1 expression resulted in a decrease of DNA repair in response to radiation. Other in vitro evidence reported inhibition of SIRT1 expression increased the efficacy of radiation against human lung cancer cells and lack of SIRT1 increased cell sensitivity to radiation. The relationship between SIRT1 and cisplatin has also been investigated in vitro. SIRT1 appears to be part of the cellular response to cisplatin, with greater SIRT1 expression associated with increased resistance of cancer cells to this treatment. Conversely, interfering with SIRT1 expression sensitized cells to cisplatin. SIRT1- and SIRT2 deficient cells were also reportedly more sensitive to the pro-apoptotic effects of cisplatin and staurosporine. This evidence, although in vitro and limited, suggests there might be interactions with the sirtuin response and certain cancer therapies that might interfere with or mitigate the efficacy of these therapies.

### Blood Coagulation Disorders

In other aspects, sirtuin-modulating compounds that increase the level and/or activity of a sirtuin protein can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. Further, the formation of blood clots does not only limit bleeding in case of an injury (hemostasis), but may lead to serious organ damage and death in the context of atherosclerotic diseases by occlusion of an important artery or vein.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism. As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis. In one aspect, the invention provides a method for reducing or inhibiting hemostasis in a subject by administering a sirtuin-modulating compound that increases the level and/or activity of a sirtuin protein. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. In another embodiment, a combination drug regimen may include drugs or compounds for the treatment or prevention of blood coagulation disorders or secondary conditions associated with these conditions. Thus, a combination drug regimen may include one or more sirtuin-modulating compounds that increase the level and/or activity of a sirtuin protein and one or more anti-coagulation or anti-thrombosis agents.

### Inflammatory Diseases

In other aspects, sirtuin-modulating compounds that increase the level and/or activity of a sirtuin protein can be used to treat or prevent a disease or disorder associated with inflammation. Sirtuin-modulating compounds that increase the level and/or activity of a sirtuin protein may be administered prior to the onset of, at, or after the initiation of inflammation. When used prophylactically, the compounds are preferably provided in advance of any inflammatory response or symptom. Administration of the compounds may prevent or attenuate inflammatory responses or symptoms. NF-κB is a transcription factor that mediates immune responses and inflammation, and it is known that NF-κB activation is an inflammatory target. The SIRT1 modulators of the present invention that deacetylate the p65/RelA subunit of NE-KB may thus suppress stimuli-induced NE-KB activation and could therefore play a role in inflammation control and modulation of the immune response.

In another embodiment, sirtuin-modulating compounds that increase the level and/or activity of a sirtuin protein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C. Additionally, sirtuin-modulating compounds that increase the level and/or activity of a sirtuin protein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more sirtuin-modulating compounds that increase the level and/or activity of a sirtuin protein may be taken alone or in combination with other compounds useful for treating or preventing inflammation.

### Ocular Disorders

One aspect of the present invention is a method for inhibiting, reducing or otherwise treating vision impairment by administering to a patient a therapeutic dosage of sirtuin modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis. In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disruption of the macula (e.g., exudative or non-exudative macular degeneration). Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes. Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a sirtuin modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a sirtuin modulator disclosed herein. Ocular surgeries include cataract, iridotomy and lens replacements.

Another aspect of the invention is the treatment, including inhibition and prophylactic treatment, of age related ocular diseases include cataracts, dry eye, age-related macular degeneration (AMD), retinal damage and the like, by administering to the subject in need of such treatment a therapeutic dosage of a sirtuin modulator disclosed herein.

Another aspect of the invention is the prevention or treatment of damage to the eye caused by stress, chemical insult or radiation, by administering to the subject in need of such treatment a therapeutic dosage of a sirtuin modulator disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more sirtuin activators and one or more therapeutic agents for the treatment of an ocular disorder.

In one embodiment, a sirtuin modulator can be administered in conjunction with a therapy for reducing intraocular pressure. In another embodiment, a sirtuin modulator can be administered in conjunction with a therapy for treating and/or preventing glaucoma. In yet another embodiment, a sirtuin modulator can be administered in conjunction with a therapy for treating and/or preventing optic neuritis. In one embodiment, a sirtuin modulator can be administered in conjunction with a therapy for treating and/or preventing CMV Retinopathy. In another embodiment, a sirtuin modulator can be administered in conjunction with a therapy for treating and/or preventing multiple sclerosis.

### Mitochondrial-Associated Diseases and Disorders

In certain embodiments, the invention provides methods for treating diseases or disorders that would benefit from increased mitochondrial activity. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular, genetic, pathologic and/or biochemical analyses. Diseases and disorders associated with mitochondrial dysfunction include diseases and disorders in which deficits in mitochondrial respiratory chain activity contribute to the development of pathophysiology of such diseases or disorders in a mammal. Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction or an agent useful for reducing a symptom associated with a disease or disorder involving mitochondrial dysfunction. In exemplary embodiments, the invention provides methods for treating diseases or disorders that would benefit from increased mitochondrial activity by administering to a subject a therapeutically effective amount of a sirtuin activating compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migraine, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation. Muscular dystrophy refers to a family of diseases involving deterioration of neuromuscular structure and function, often resulting in atrophy of skeletal muscle and myocardial dysfunction, such as Duchenne muscular dystrophy. In certain embodiments, sirtuin activating compounds may be used for reducing the rate of decline in muscular functional capacities and for improving muscular functional status in patients with muscular dystrophy. In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal).

In certain embodiments, sirtuin activating compounds may be useful for treating patients suffering from toxic damage to mitochondria, such as, toxic damage due to calcium accumulation, excitotoxicity, nitric oxide exposure, drug induced toxic damage, or hypoxia. In certain embodiments, sirtuin activating compounds may be useful for treating diseases or disorders associated with mitochondrial deregulation.

### Muscle Performance

In other embodiments, the invention provides methods for enhancing muscle performance by administering a therapeutically effective amount of a sirtuin activating compound. For example, sirtuin activating compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a sirtuin activating compound that increase mitochondrial activity, increase mitochondrial biogenesis, and/or increase mitochondrial mass.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time.

It is contemplated that the methods of the present invention will also be effective in the treatment of muscle related pathological conditions, including acute sarcopenia, for example, muscle atrophy and/or cachexia associated with burns, bed rest, limb immobilization, or major thoracic, abdominal, and/or orthopedic surgery. In certain embodiments, the invention provides novel dietary compositions comprising sirtuin modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

### Flushing

In another aspect, sirtuin-modulating compounds that increase the level and/or activity of a sirtuin protein may be used for reducing the incidence or severity of flushing and/or hot flashes which are symptoms of a disorder or arises as a side effect from treatment with other agents. In other embodiments, the method provides for the use of sirtuin-modulating compounds that increase the level and/or activity of a sirtuin protein to reduce the incidence or severity of flushing and/or hot flashes in menopausal and post-menopausal woman.

### Pulmonary Disease

In still another aspect, sirtuin-modulating compounds, such as the novel compounds of the invention that increase the level and/or activity of a sirtuin protein may be used for the treatment of certain lung diseases. A recently published study by Yao H et al. (J Clin Invest. 2012 Jun. 1; 122(6):2032-45) showed that chronic obstructive pulmonary disease/emphysema (COPD/emphysema) is characterized by chronic inflammation and premature lung aging. The anti-aging sirtuin 1 (SIRT1) is reduced in lungs of patients with COPD. However, the molecular signals underlying the premature aging in lungs, and whether SIRT1 protects against cellular senescence and various pathophysiological alterations in emphysema, remain unknown. The study showed increased cellular senescence in lungs of COPD patients. SIRT1 activation by both genetic overexpression and a selective pharmacological activator, SRT1720, attenuated stress-induced premature cellular senescence and protected against emphysema induced by cigarette smoke and elastase in mice. Ablation of Sirt1 in airway epithelium, but not in myeloid cells, aggravated airspace enlargement, impaired lung function, and reduced exercise tolerance. These effects were due to the ability of SIRT1 to deacetylate the FOXO3 transcription factor, since Foxo3 deficiency diminished the protective effect of SRT1720 on cellular senescence and emphysematous changes. Thus, SIRT1 protects against emphysema through FOXO3-mediated reduction of cellular senescence, independently of inflammation. Thus, activation of SIRT1 may be an attractive therapeutic strategy in COPD/emphysema using the Sirt1 activators of the invention.

### CONCLUSIONS

As research has better characterized the sirtuin system, it has become apparent that this system regulates many proteins, which themselves influence a variety of cellular processes. Because of their impact on the function of a diverse array of proteins, sirtuins are involved with metabolic responses and processes that influence many aspects of human function. Existing evidence strongly supports sirtuin involvement in longevity, age-related diseases, obesity, cardiovascular and neurological function and cancer. As these responses become better understood, which sirtuins to target for activation or inhibition should become clearer. Cancer is a good example. Experimental evidence argues that sirtuins play a complex, more nuanced role in cancer than can be determined by its effects on any protein or metabolic process viewed in isolation. The complicated and perhaps competing effects of individual sirtuins on cellular processes that influence cancer development, suppression, and progression suggest much more research is required. Although SIRT1 has been found to increase in some cancers and not in others, its increase alone cannot be taken as evidence that it is a cause of cancer development. On the other hand, it could be a consequence of tumorigenesis or other factors involved in cancer or an adaptive response intended to counter genotoxic insults that contribute to cancer. Although sirtuin expression might counteract the desired clinical response to certain cancer therapies, specifically radiation and chemotherapy, there might be times when an increased sirtuin response might enhance cancer prevention or treatment. Currently there are as many questions as there are answers.

Sirtuin-modulating compounds that increase the level and/or activity of a sirtuin protein may be used for treating or preventing viral infections (such as infections by influenza, herpes or papilloma virus) or as antifungal agents. In certain embodiments, sirtuin-modulating compounds that increase the level and/or activity of a sirtuin protein may be administered as part of a combination drug therapy with another therapeutic agent for the treatment of viral diseases. In another embodiment, sirtuin-modulating compounds that increase the level and/or activity of a sirtuin protein may be administered as part of a combination drug therapy with another antifungal agent. Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells.

Generally, the methods described herein may be applied to any organism, e.g., eukaryote, that may have commercial importance. For example, they can be applied to fish (aquaculture) and birds (e.g., chicken and fowl).

### Assays

Various types of assays to determine sirtuin activity have been described. For example, sirtuin activity may be determined using a fluorescence based assay such as the assay commercially available from Cisbio or from Biomol, e.g., the SIRT1 Fluorimetric Drug Discovery Kit (AK-555), SIRT2 Fluorimetric Drug Discovery Kit (AK-556), or SIRT3 Fluorimetric Drug Discovery Kit (AK-557) (Biomol International, Plymouth Meeting, Pa.). Other suitable sirtuin assays include a nicotinamide release assay (Kaeberlein et al., J. Biol. Chem. 280(17): 17038 (2005)), a FRET assay (Marcotte et al., Anal. Biochem. 332: 90 (2004)), and a C¹⁴ NAD boron resin binding assay (McDonagh et al., Methods 36: 346 (2005)). Robers M. B. et al describe the measurement of the cellular deacetylase activity of SIRT1 on p53 via LanthaScreen® technology.

Yet other suitable sirtuin assays include radioimmunoassays (RIA), scintillation proximity assays, HPLC based assays, and reporter gene assays (e.g., for transcription factor targets). An exemplary assay for determining sirtuin activity is a fluorescence polarization assay. Fluorescence polarization assays are described herein and are also described in PCT Publication No. WO 2006/094239. In other embodiments, sirtuin activity may be determined using a mass spectrometry based assays. Examples of mass spectrometry based assays are described herein and are also described in PCT Publication No. WO 2007/064902. Cell based assays may also be used to determine sirtuin activity. Examples of cell based assays for determining sirtuin activity are described in PCT Publication Nos. WO 2007/064902 and WO 2008/060400. Yet other methods contemplated herein include screening methods for identifying compounds or agents that modulate sirtuins. An agent may be a nucleic acid, such as an aptamer. Assays may be conducted in a cell based or cell free format. For example, an assay may comprise incubating (or contacting) a sirtuin with a test agent under conditions in which a sirtuin can be modulated by an agent known to modulate the sirtuin, and monitoring or determining the level of modulation of the sirtuin in the presence of the test agent relative to the absence of the test agent. The level of modulation of a sirtuin can be determined by determining its ability to deacetylate a substrate. Exemplary substrates are acetylated peptides which can be obtained from BIOMOL (Plymouth Meeting, Pa.). Preferred substrates include peptides of p53, such as those comprising an acetylated K382. A particularly preferred substrate is the Fluor de Lys-SIRT1 (BIOMOL), i.e., the acetylated peptide Arg-His-Lys-Lys. Other substrates are peptides from human histones H3 and H4 or an acetylated amino acid. Substrates may be fluorogenic. The sirtuin may be SIRT1, Sir2, SIRT3, or a portion thereof. For example, recombinant SIRT1 can be obtained from BIOMOL. The reaction may be conducted for about 30 minutes and stopped, e.g., with nicotinamide. The HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of modulation of the sirtuin in an assay may be compared to the level of modulation of the sirtuin in the presence of one or more (separately or simultaneously) compounds described herein, which may serve as positive or negative controls. Sirtuins for use in the assays may be full length sirtuin proteins or portions thereof. Since it has been shown herein that activating compounds appear to interact with the N-terminus of SIRT1, proteins for use in the assays include N-teπninal portions of sirtuins, e.g., about amino acids 1-176 or 1-255 of SIRT1; about amino acids 1-174 or 1-252 of Sir2.

In one embodiment, a screening assay comprises (i) contacting a sirtuin with a test agent and an acetylated substrate under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin. Methods for identifying an agent that modulates, e.g., stimulates, sirtuins in vivo may comprise (i) contacting a cell with a test agent and a substrate that is capable of entering a cell in the presence of an inhibitor of class I and class II HDACs under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin. A preferred substrate is an acetylated peptide, which is also preferably fluorogenic, as further described herein. The method may further comprise lysing the cells to determine the level of acetylation of the substrate.

Substrates may be added to cells at a concentration ranging from about 1 μM to about 10 mM, preferably from about 10 μM to about 1 mM, even more preferably from about 100 μM to about 1 mM, such as about 200 μM. A preferred substrate is an acetylated lysine, e.g., E-acetyl lysine (Fluor de Lys, FdL) or Fluor de Lys-SIRT1. A preferred inhibitor of class I and class II HDACs is trichostatin A (TSA), which may be used at concentrations ranging from about 0.01 to 100 μM, preferably from about 0.1 to 10 μM, such as 1 μM. Incubation of cells with the test compound and the substrate may be conducted for about 10 minutes to 5 hours, preferably for about 1-3 hours. Since TSA inhibits all class I and class II HDACs, and that certain substrates, e.g., Fluor de Lys, is a poor substrate for SIRT2 and even less a substrate for SIRT3-7, such an assay may be used to identify modulators of SIRT1 in vivo.

### Pharmacological Activity and Availability

The compounds of the invention are pharmacologically active compounds or substances, which may act both systemically and/or locally in a subject. Basically, the four parameters of importance in determining whether a chemical compound is pharmacologically active or bioavailable are generally accepted in the art as the “Lipinski's rules”: a molecular weight below 500 Daltons; a limited lipophilicity, e.g. as expressed by Log P<5 with P=[drug]_{org}/[drug]_{aq}.; a maximum of 5H-bond donors, e.g. expressed as the sum of OHs and NHs; and a maximum of 10H-bond acceptors, e.g. expressed as the sum of oxygen atoms and nitrogen atoms. The Lipinski rules are fulfilled by a broad range of the compounds of the invention, cf. i.a., Table 4 below and the structural formulae I to IV.

### Examples of Pharmaceutical Compositions

The sirtuin-modulating compounds described herein may be formulated in a conventional manner using one or more physiologically acceptable carriers or excipients. For example, sirtuin-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a sirtuin-modulating compound may be administered locally, at the site where the target cells are present, i.e., in a specific tissue, organ, or fluid (e.g., blood, cerebrospinal fluid, etc.). Sirtuin-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound. For administration by inhalation (e.g., pulmonary delivery), sirtuin-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch. Sirtuin-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. Sirtuin-modulating compounds may also be formulated in rectal compositions such as suppositories or retention enemas, e.g., containing conventional suppository bases such as cocoa butter or other glycerides.

In addition to the formulations described previously, sirtuin-modulating compounds may also be formulated as a depot preparation. Such long acting formulations may be administered by implantation (for example subcutaneously or intramuscularly) or by intramuscular injection. Thus, for example, sirtuin-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidyl-cholines. Liposomes and polymersomes being usable for the method of invention encompass all types including, but not limited to, small unilamellar vesicles, large unilamellar vesicles and multilamellar vesicles.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent. Pharmaceutical compositions (including cosmetic preparations) may comprise from about 0.00001 to 100% such as from 0.001 to 10% or from 0.1% to 5% by weight of one or more sirtuin-modulating compounds described herein.

In one embodiment, a sirtuin-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. Examples of suitable topical carriers for use herein include water, alcohols and other nontoxic organic solvents, glycerin, mineral oil, silicone, petroleum jelly, lanolin, fatty acids, vegetable oils, parabenes, waxes, and the like. Formulations may be colorless, odorless ointments, lotions, creams, microemulsions and gels. Other active agents may also be included in formulations, e.g., other antiinflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. We suggest that conditions of the eye, such as age related cataract, can be treated or prevented by, e.g., systemic, topical, intraocular injection of a sirtuin-modulating compound, or by insertion of a sustained release device that releases a sirtuin-modulating compound, cf. T. J. Lin et al. (2011), who have found that the decreased expression of SirT1 in the lens epithelium was associated with higher cataract scores and patient age. The results suggest that a local SirT1 decrease in cataractous lens could be a risk factor for the initiation of age-related cataract formation. A sirtuin-modulating compound that increases the level and/or activity of a sirtuin protein may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

Sirtuin-modulating compounds described herein may be stored in oxygen free environment. Cells, e.g., treated ex vivo with a sirtuin-modulating compound, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

The data obtained from the cell culture assays and animal studies can be used in formulating a range of dosage for use in humans. The dosage of such compounds may lie within a range of circulating concentrations that include the ED50 with lithe or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Kits provided herein are kits, e.g., kits for therapeutic purposes or kits for modulating the lifespan of cells or modulating apoptosis. A kit may comprise one or more sirtuin-modulating compounds, e.g., in premeasured doses. A kit may optionally comprise devices for contacting cells with the compounds and instructions for use. Devices include syringes, stents and other devices for introducing a sirtuin-modulating compound into a subject (e.g., the blood vessel of a subject) or applying it to the skin of a subject.

In yet another embodiment, the invention provides a composition of matter comprising a sirtruin modulator of this invention and another therapeutic agent (the same ones used in combination therapies and combination compositions) in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The agent and the sirtruin modulator are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a sirtuin modulator of this invention; and b) another another therapeutic agent such as those described elsewhere in the specification.

Additionally, the invention relates in a broader aspect to compounds of Formula II and IIa:

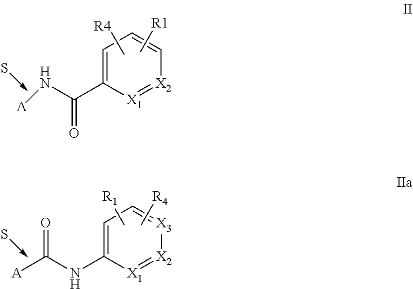

wherein X₁ and X₂ are as defined in relation to Formula I, above, or one of X₁ and X₂ is missing resulting in a pyrrole ring;
R1 is a substituent selected from —C(═O)—R5, —C(═O)—N(CH₃)₂, —C(═O)—NHCH₂, —C(═S)—R5, —N—C(═O)—R5, —S(═O)₂—R5, or —S(═O)—R5,

R4 is an optional substituent selected from lower alkyl such as methyl; s represents a single covalent bond, a linear oligomethylene group, or a methylene group, and then A is an aromatic ring system selected from pyrrole, pyridine, phenyl, naphthyl including 1-naphtyl and 2-naphthyl, quinoline including all isomers, isoquinoline including all isomers, indole including all isomers, isoindole including all isomers, and wherein A has at least one nucleophilic substituent such as at least one hydroxyl group or primary amino group; A optionally has one or more other substituents selected from cyano, NO₂, OH, a halogen such as F, Cl and Br; —C(═S)—R5, —C(═O)—R5; —N—C(═O)—R5, —S(═O)₂—R5, and —S(═O)—R5; or

s is absent and the group A represents an aromatic heterocyclic ring system selected from pyrrol, indole, and isoindole wherein the heteroatomic nitrogen replaces the amide nitrogen of the compounds of formula II, and wherein A has at least one nucleophilic substituent such as at least one hydroxyl group or primary amino group; A optionally has one or more other substituents selected from cyano, NO₂, OH, a halogen such as F, Cl and Br; —C(═S)—R5, —C(═O)—R5; —N—C(═O)—R5, —S(═O)₂—R5, and —S(═O)—R5; with the exception that s does not represent a covalent bond in Formula IIa.

R5 is NH₂ optionally substituted with C₁₋₄ lower alkyl, or R5 is CH₃;

and solvates, tautomers or isomers thereof including pharmaceutically acceptable salts, acid addition salts and base addition salts. In addition, heterocyclic compounds having an analogous chemical structure as the compounds of formula I, but wherein the central amide functionality is inverted, are also included in the present invention. In these compounds the various substituents including A are as defined above.

Preferred compounds of formula II or IIa are selected from the group consisting of formulae III, IIIa, IV and IVa:

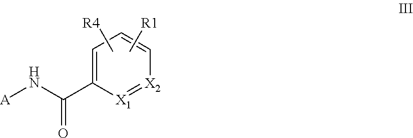

### Formula IIIa:

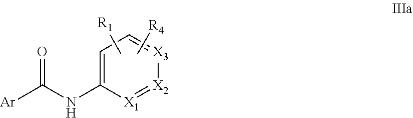

wherein X1, X2, R1, R4 and A are as defined above,

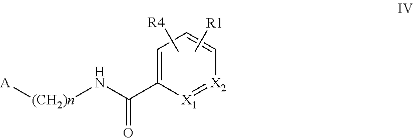

### Formula IVa:

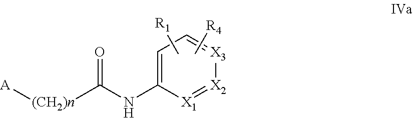

wherein X1, X2, R1, R4 and A are as defined in claim 1, and n is an integer selected from 0, 1 and 2; and solvates, tautomers or isomers thereof including pharmaceutically acceptable salts, acid addition salts and base addition salts.

The compounds of formulae II, III, and IV may be prepared according to general synthetic scheme c):

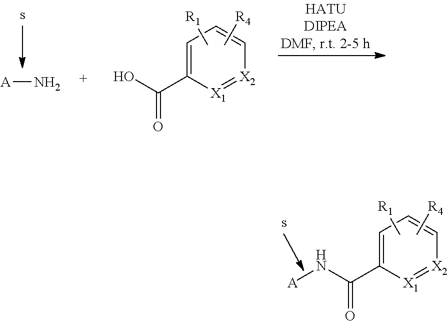

Wherein HATU, DIPEA, and DMF are as previously disclosed, and the reaction may take place in solution phase or solid phase as will be generally known by the skilled person in the art.

Moreover, the compounds of formulae IIa, IIIa, and IVa may be prepared according to general synthetic scheme d):

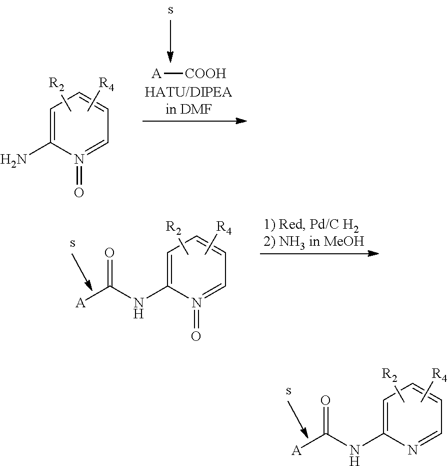

with the exception of compounds wherein s represents a covalent bond.

Exemplary compounds of the invention are listed in Table 1 below:

IUPAC name

2-N(4-hydroxyphenyl)pyridine-2,5-dicarboxamide
2-N(2-hydroxyphenyl)pyridine-2,5-dicarboxamide
5-N(2-hydroxyphenyl)pyridine-2,5-dicarboxamide
5-N(4-hydroxyphenyl)pyridine-2,5-dicarboxamide
2-N(2,4-dihydroxyphenyl)pyridine-2,5-dicarboxamide
2-N(2,4-dihydroxyphenyl)pyridine-2,6-dicarboxamide
5-N(2,4-dihydroxyphenyl)pyridine-2,5-dicarboxamide
2-N(5-hydroxy-1-naphthyl)pyridine-2,5-dicarboxamide
2-N(6-hydroxy-1-naphthyl)pyridine-2,5-dicarboxamide
2-N(4-hydroxy-1-naphthyl)pyridine-2,5-dicarboxamide
5-N(5-hydroxy-1-naphthyl)pyridine-2,5-dicarboxamide
5-N(6-hydroxy-1-naphthyl)pyridine-2,5-dicarboxamide
5-N(4-hydroxy-1-naphthyl)pyridine-2,5-dicarboxamide
6-N(4-hydroxyphenyl)quinolin-2,6-dicarboxamide
6-N(2-hydroxyphenyl)quinolin-2,6-dicarboxamide
4-N(4-hydroxyphenyl)pyrrol-2,4-dicarboxamide
4-N(2-hydroxyphenyl)pyrrol-2,4-dicarboxamide

Further exemplary compounds are listed below:
2-N(4-hydroxyphenyl)pyridine-2,5-dicarboxamides:
2-N(3-chloro-4-hydroxyphenyl)pyridine-2,5-dicarboxamide
2-N(3-fluoro-4-hydroxyphenyl)pyridine-2,5-dicarboxamide
2-N(3-cyano-4-hydroxyphenyl)pyridine-2,5-dicarboxamide
2-N(3-carboxamido-4-hydroxyphenyl)pyridine-2,5-dicarboxamide
2-N(2-chloro-4-hydroxyphenyl)pyridine-2,5-dicarboxamide
2-N(2-fluoro-4-hydroxyphenyl)pyridine-2,5-dicarboxamide
2-N(2-cyano-4-hydroxyphenyl)pyridine-2,5-dicarboxamide
2-N(2-carboxamido-4-hydroxyphenyl)pyridine-2,5-dicarboxamide
2-N(2-hydroxyphenyl)pyridine-2,5-dicarboxamides:
2-N(3-chloro-2-hydroxyphenyl)pyridine-2,5-dicarboxamide
2-N(3-fluoro-2-hydroxyphenyl)pyridine-2,5-dicarboxamide
2-N(3-cyano-2-hydroxyphenyl)pyridine-2,5-dicarboxamide
2-N(3-carboxamido-2-hydroxyphenyl)pyridine-2,5-dicarboxamide
2-N(4-chloro-2-hydroxyphenyl)pyridine-2,5-dicarboxamide
2-N(4-fluoro-2-hydroxyphenyl)pyridine-2,5-dicarboxamide
2-N(4-cyano-2-hydroxyphenyl)pyridine-2,5-dicarboxamide
2-N(4-carboxamido-2-hydroxyphenyl)pyridine-2,5-dicarboxamide
2-N(6-chloro-2-hydroxyphenyl)pyridine-2,5-dicarboxamide
2-N(6-fluoro-2-hydroxyphenyl)pyridine-2,5-dicarboxamide
2-N(6-cyano-2-hydroxyphenyl)pyridine-2,5-dicarboxamide
2-N(6-carboxamido-2-hydroxyphenyl)pyridine-2,5-dicarboxamide
2-N(5-chloro-2-hydroxyphenyl)pyridine-2,5-dicarboxamide
2-N(5-fluoro-2-hydroxyphenyl)pyridine-2,5-dicarboxamide
2-N(5-cyano-2-hydroxyphenyl)pyridine-2,5-dicarboxamide
2-N(5-carboxamido-2-hydroxyphenyl)pyridine-2,5-dicarboxamide
5-N(4-hydroxyphenyl)pyridine-2,5-dicarboxamides:
5-N(3-chloro-4-hydroxyphenyl)pyridine-2,5-dicarboxamide
5-N(3-fluoro-4-hydroxyphenyl)pyridine-2,5-dicarboxamide
5-N(3-cyano-4-hydroxyphenyl)pyridine-2,5-dicarboxamide
5-N(3-carboxamido-4-hydroxyphenyl)pyridine-2,5-dicarboxamide
5-N(2-chloro-4-hydroxyphenyl)pyridine-2,5-dicarboxamide
5-N(2-fluoro-4-hydroxyphenyl)pyridine-2,5-dicarboxamide
5-N(2-cyano-4-hydroxyphenyl)pyridine-2,5-dicarboxamide
5-N(2-carboxamido-4-hydroxyphenyl)pyridine-2,5-dicarboxamide
5-N(2-hydroxyphenyl)pyridine-2,5-dicarboxamides:
5-N(3-chloro-2-hydroxyphenyl)pyridine-2,5-dicarboxamide
5-N(3-fluoro-2-hydroxyphenyl)pyridine-2,5-dicarboxamide
5-N(3-cyano-2-hydroxyphenyl)pyridine-2,5-dicarboxamide
5-N(3-carboxamido-2-hydroxyphenyl)pyridine-2,5-dicarboxamide
5-N(4-chloro-2-hydroxyphenyl)pyridine-2,5-dicarboxamide
5-N(4-fluoro-2-hydroxyphenyl)pyridine-2,5-dicarboxamide
5-N(4-cyano-2-hydroxyphenyl)pyridine-2,5-dicarboxamide
5-N(4-carboxamido-2-hydroxyphenyl)pyridine-2,5-dicarboxamide
5-N(6-chloro-2-hydroxyphenyl)pyridine-2,5-dicarboxamide
5-N(6-fluoro-2-hydroxyphenyl)pyridine-2,5-dicarboxamide
5-N(6-cyano-2-hydroxyphenyl)pyridine-2,5-dicarboxamide
5-N(6-carboxamido-2-hydroxyphenyl)pyridine-2,5-dicarboxamide
5-N(5-chloro-2-hydroxyphenyl)pyridine-2,5-dicarboxamide
5-N(5-fluoro-2-hydroxyphenyl)pyridine-2,5-dicarboxamide
5-N(5-cyano-2-hydroxyphenyl)pyridine-2,5-dicarboxamide
5-N(5-carboxamido-2-hydroxyphenyl)pyridine-2,5-dicarboxamide

### EXPERIMENTAL SECTION, EXAMPLES, ASPECTS AND EMBODIMENTS OF THE INVENTION

### Example 1

### Preparation of the Substituted Pyridine Dicarboxamides of the Invention

### General Synthetic Procedure:

### Amide Formation Based on HATU Coupling

A solution of the pyridine 2-carboxylic acid, 5-carboxamide (1 eq, compound shown to the left in reaction scheme 1 below), HATU (2-(1H-7-Azabenzotriazol-1-yl)-1,1,3,3-tetramethyl uronium hexafluorophosphate methanaminium, 1.3 eq) and diisopropylethylamine (DIPEA, 2.5 eq) in 20 ml dimethylformamide (DMF) is stirred at room temperature for 30 minutes. After stirring the reaction mixture for 30 minutes, the R—NH2 amine (1 eq, or slight excess, where R represents the aromatic ring system Ar and A defined above and in the claims) was added and stirred for 2-3 hours. The resulting mixture was diluted with 20-50 ml ethyl acetate and washed with aqueous 1N HCl (2×), brine (3×), dried over MgSO₄, filtered and concentrated to give the crude dicarboxamide, e.g. compounds 1-6, 18-24, and 27-32. This crude material was purified using preparative HPLC, buffer system A: water with 0.1% TFA B: 90% acetonitrile/9.9% Water/0.1% TFA. In the synthesis of compounds 7-12 a pyridine 5-carboxylic acid, 3-carboxamide is used as the starting compound. In the synthesis of compound 13 a pyridine 2-carboxylic acid, 6-carboxamide is used as the starting compound.

The reaction generally follows scheme 1 shown below.

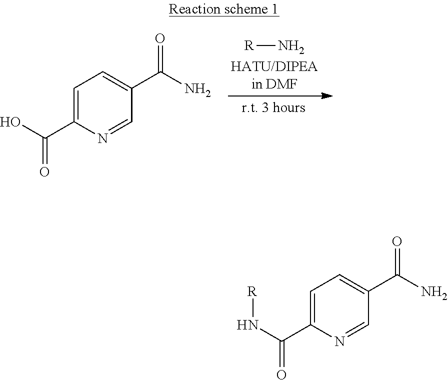

### Alternatively, the Compounds May be Synthesized by Amide Formation Based on Acid Chloride Coupling:

Triethylamine (1.5 equiv), and the amine (1.30 equiv) is dissolved in DCM and the mixture is cooled to an internal temperature of <5° C. 1.3 eq. acid chloride is dissolved in DCM and added drop wise to the stirred amine solution. Upon completion of the addition of the acid chloride solution, the mixture is stirred at room temperature for 30 min. The mixture is then diluted with 2 N HCl and the layers separated. The organic layer is washed with brine and is then concentrated under reduced pressure (23° C., 40 mmHg), diluted with MeOH and re-concentrated under reduced pressure (23° C., 40 mmHg) to give a crude product.

The residue was dissolved in a saturated methanolic NH3 solution (15 mL) at room temperature and placed in a sealed tube and the temperature was raised slowly to 70° C. for 30 min. The solvent was evaporated and the residue was purified using preparative HPLC.

The reaction generally follows scheme 2 shown below.

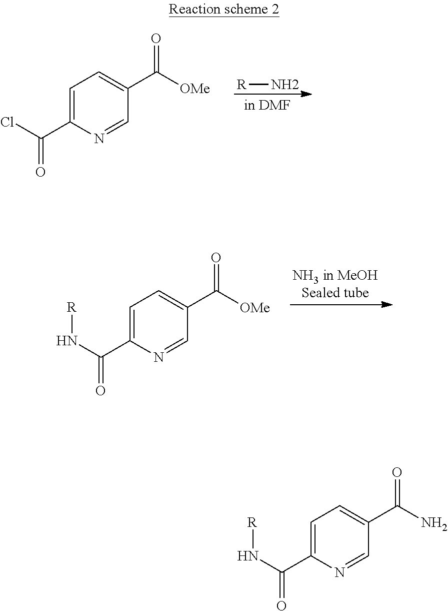

### Amide Formation Via 2-Amino-Pyridine N-Oxides, Synthesis of Compounds 25 and 26.

The 2-amino-pyridine N-oxide was coupled to the carboxylic acid R—COOH, where R represents the aromatic ring system Ar or A defined above and in the claims, resulting in the corresponding amide according to the procedure outlined above scheme 2 and 3. The resulting N-oxide amide product was subsequently dissolved in methanol at room temperature and reduced using hydrogen (>1 atm.) and PD/C for 14 h. The solution was filtered and evaporated. The residue was purified using HPLC.

The reaction generally follows scheme 3 shown below.

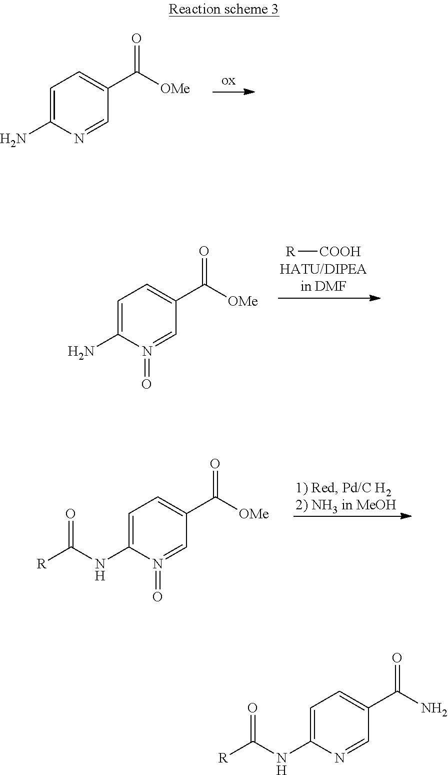

Average molecular weights and calculated molecular weights and molecular weights found from mass spectroscopic analysis are given in Table 2 below:

TABLE 2

| | | | Exact Mw | Exact Mw |
|---|---|---|---|---|
| Cpd No. | IUPAC name of compound | MW average | calc | found |
| 1 | 2-N(4-hydroxyphenyl)pyridine-2,5- | 257.24 | 257.08 | 257.11 |
| | dicarboxamide | | | |
| 2 | 2-N(2-hydroxyphenyl)pyridine-2,5- | 257.24 | 257.08 | 257.09 |
| | dicarboxamide | | | |
| 3 | 2-N(2,4-dihydroxyphenyl)pyridine-2,5- | 273.24 | 273.07 | 273.12 |
| | dicarboxamide | | | |
| 4 | 2-N(4-hydroxy-1-naphthyl)pyridine-2,5- | 307.30 | 307.10 | 307.08 |
| | dicarboxamide | | | |
| 5 | 2-N(5-hydroxy-1-naphthyl)pyridine-2,5- | 307.30 | 307.10 | 307.14 |
| | dicarboxamide | | | |
| 6 | 2-N(6-hydroxy-1-naphthyl)pyridine-2,5- | 307.30 | 307.10 | 307.09 |
| | dicarboxamide | | | |
| 7 | 5-N(4-hydroxyphenyl)pyridine-2,5- | 257.24 | 257.08 | 257.11 |
| | dicarboxamide | | | |
| 8 | 5-N(2-hydroxyphenyl)pyridine-2,5- | 257.24 | 257.08 | 257.07 |
| | dicarboxamide | | | |
| 9 | 5-N(2,4-dihydroxyphenyl)pyridine-2,5- | 273.24 | 273.07 | 273.11 |
| | dicarboxamide | | | |
| 10 | 5-N(5-hydroxy-1-naphthyl)pyridine-2,5- | 307.30 | 307.10 | 307.15 |
| | dicarboxamide | | | |
| 11 | 5-N(4-hydroxy-1-naphthyl)pyridine-2,5- | 307.30 | 307.10 | 307.11 |
| | dicarboxamide | | | |
| 12 | 5-N(6-hydroxy-1-naphthyl)pyridine-2,5- | 307.30 | 307.10 | 307.13 |
| | dicarboxamide | | | |
| 13 | 2-N(2,4-dihydroxyphenyl)pyridine-2,6- | 273.24 | 273.07 | 273.09 |
| | dicarboxamide | | | |
| 14 | 6-N(4-hydroxyphenyl)quinolin-2,6- | 307.30 | 307.10 | 307.15 |
| | dicarboxamide | | | |
| 15 | 6-N(2-hydroxyphenyl)quinolin-2,6- | 307.30 | 307.10 | 307.17 |
| | dicarboxamide | | | |
| 16 | 4-N(4-hydroxyphenyl)pyrrol-2,4- | 245.23 | 245.08 | 245.09 |
| | dicarboxamide | | | |
| 17 | 4-N(2-hydroxyphenyl)pyrrol-2,4- | 245.23 | 245.08 | 245.12 |
| | dicarboxamide | | | |

Additional compounds are listed in the table below

| Comp. | |
|---|---|
| No | IUPAC name |
| 18 | 2-N(3-hydroxy-2-naphthyl)pyridine-2,5-dicarboxamide |
| 19 | 2-N(1-hydroxy-2-naphthyl)pyridine-2,5-dicarboxamide |
| 20 | 2-N(2-hydroxy-1-naphthyl)pyridine-2,5-dicarboxamide |
| 21 | 2-N(2-hydroxy-3-chloro-phenyl)pyridine-2,5-dicarboxamide |
| 22 | 2-N(2-hydroxy-5-methoxy-phenyl)pyridine-2,5-dicarboxamide |
| 23 | 2-N(2-hydroxy-5-chloro-phenyl)pyridine-2,5-dicarboxamide |
| 24 | 2-N(2-hydroxy-3-methoxy-phenyl)pyridine-2,5-dicarboxamide |
| 25 | 6-(2-hydroxy-benzoylamino)-pyridine-3-carboxamide |
| 26 | 6-(2-hydroxy-1-naphthoylamino)-pyridine-3-carboxamide |
| 27 | 2-N(2-hydroxy-6-methoxy-1-naphthyl)pyridine-2,5- |
| | dicarboxamide |
| 28 | 2-N(2-hydroxy-3-methoxy-1-naphthyl)pyridine-2,5- |
| | dicarboxamide |
| 29 | 2-N(2-hydroxy-6-chloro-1-naphthyl)pyridine-2,5-dicarboxamide |
| 30 | 2-N(2-hydroxy-3-chloro-1-naphthyl)pyridine-2,5-dicarboxamide |
| 31 | 2-N(2-hydroxy-8-methoxy-1-naphthyl)pyridine-2,5- |
| | dicarboxamide |
| 32 | 2-N(2-hydroxy-8-chloro-1-naphthyl)pyridine-2,5-dicarboxamide |
| 34 | 3-N(4-hydroxynaphthalene-1-yl)Pyridine-3,5-dicarboxamide |
| 36 | 5-N(3,4-dihydroxynaphthalene-1-yl)Pyridine-2,5-dicarboxamide |
| 37 | 5-N(4,5-dihydroxynaphthalene-1-yl)Pyridine-2,5-dicarboxamide |
| 38 | 5-N(4-hydroxynaphthalene-1-ylmethyl)Pyridine-2,5- |
| | dicarboxamide |

The net weights in mg, av. molecular weights, and purity obtained after synthesis according to HPLC analysis of some of the novel compounds are shown in Table 3 below:

TABLE 3

| | Yield Net Weight | | |
|---|---|---|---|
| Cmpd No. | (mg) | Molecular Weight | Purity % |
| 1 | 125 | 257.24 | 95.5 |
| 2 | 112 | 257.24 | 95.4 |
| 3 | 109 | 273.24 | 95.1 |
| 4 | 170 | 307.30 | 99.8 |
| 5 | 44 | 307.30 | 91.6 |
| 6 | 58 | 307.30 | 97.8 |
| 7 | 53 | 257.24 | 97.3 |
| 8 | 54 | 257.24 | 99.7 |
| 9 | 60 | 273.24 | 95.4 |
| 10 | 96 | 307.30 | 97.3 |
| 11 | 110 | 307.30 | 91.6 |
| 12 | 92 | 307.30 | 96.5 |
| 13 | 54 | 273.24 | 95.2 |
| 14 | 95 | 307.30 | 95.2 |
| 15 | 60 | 307.30 | 97.2 |
| 16 | 73 | 245.23 | 99.9 |
| 17 | 294 | 245.23 | 96.9 |
| 18 | 60 | 307.3 | 90 |
| 19 | 15 | 307.3 | 95 |
| 20 | 70 | 307.3 | 98 |
| 21 | 30 | 291.69 | 96 |
| 22 | 50 | 287.27 | 90 |
| 23 | 60 | 291.69 | 90 |
| 24 | 80 | 287.27 | 95 |
| 25 | 35 | 257.24 | 95 |
| 26 | 48 | 307.3 | 95 |
| 27 | 37 | 337.33 | 99 |
| 28 | 70 | 337.33 | 99 |
| 29 | 50 | 341.75 | 95 |
| 30 | 45 | 341.75 | 95 |
| 31 | 14 | 337.33 | 94 |
| 32 | 10 | 337.33 | 94 |

### Example 2

### Solubility of the Compounds of the Invention

The compounds of the invention are soluble in a DMSO stock solution for subsequent dilution with water. Table 4 below summarizes the solubilities of selected compounds of the invention in DMSO before further dilution with water to a final concentration of 1% DMSO.

TABLE 4

| Concentration of compound in DMSO stock | |
|---|---|
| solution | Compound No. |
| 200 mM | 2, 7, 9, 14, 16, 17 |
| 100 mM | 6, 8 |
| 20 mM | 3, 4, 5, 10, 11, 12, 13 |
| 10 mM | 15 |
| 5 mM | 1 |

### Example 3

### Assay Showing Modulation of SIRT1 Peptide Deacetylation Using Selected Compounds of the Invention in a CISBIO HTRF® SIRT1 Assay Kit.

Generally, the CISBIO HTRF® fluorescence enzyme titration assay (Cisbio Bioassays, Cisbio, 135 South Road, Bedford, Mass. 01730, USA) has provided information on inhibition and/or activation of SIRT1 deacetylation of a substrate peptide d2 containing a single acetylated lysine residue and a fluorescence probe/quencher. IC50 and AC50 values have been obtained. The assay was performed in microtiter wells and involved an enzymatic step, wherein 2 μL of the substrate peptide (6 nM) is incubated with 2 μL of SIRT1 enzyme (2.5 ng) and 2 μL of compound solution in DMSO and water. Reaction with anti-acetyl cryptate produces a FRET signal giving maximum signal when no SIRT1 reaction can be detected. The detection step involves quantification of the deacetylation process using an anti-acetyl MAb labeled with Eu³⁺ cryptate. The assay was modified by excluding the NAD+ cofactor and using nicotinamide as inhibition control and the compound SRT1720 as activation control. SRT 1720 is described as a SIRT1 activating compound in Pillarisetti, S. (2008).

Protocol for inhibition/activation assay:

| 10 μL enzymatic step ingredients, each | |
|---|---|
| well | 20 μL detection step |
| 4 μL substrate d2 6 nM | 10 μL Anti-acetyl MAb cryptate |
| 2 μL compound start conc. according to | 10 μL of mixed reactants |
| table 5 | |
| 2 μL enzyme; 2.5 ng | |
| 2 μL nicotinamide or SRT1720; 2.5 ng | |

After adding all enzymatic step ingredients the mixture is incubated at room temperature at 30 to 60 min. Then the detection reactant is added and incubated at room temperature for 5 hours and on to produce a signal. The fluorescence readout is given as percentage of signal produced with no enzyme reaction.

The cryptate is excited at 337 nm and the fluorescence is measured at 590 nm (cryptate emission wavelength) and 665 nm (d2 emission wavelength). A ratio is calculated (665/590 nm) for each well.

Dose response studies were made using the assay where the initial concentration of each compound of the invention was reduced with three-fold aqueous dilutions, cf. results in FIGS. 1 to 5 for the compounds 2, 9, 11, 13 and 17, respectively. The figures show percentage fluorescence as a function of Log C (log of compound concentration in μM). AC50 and IC50 values are calculated from the Log C value at 50% fluorescence. Each experiment was repeated twice to obtain an average AC50 or IC50 value as given in Table 5. Compounds 2, 9, 11, and 13 show clear activation having AC50 values in the micro molar range, whereas one compound showed clear inhibition of SIRT1 deacetylating activity in the assay: compound 17 having an IC50 value of 0.5 μM.

TABLE 5

| Compound No. | | | |
|---|---|---|---|
| and initial | AC50 in μM, | AC50 in μM, | AC50 in μM, |
| concentration | average, n = 3 | n = 1 | Exp. of Fig. No. |
| 2 2000 μM | 348 | 316 | FIG. 1 |
| 9 2000 μM | N.A. | 112 | FIG. 2 |
| 11 200 μM | 114 | 3.3 | FIG. 3 |
| 13 200 μM | 647 | 105 | FIG. 4 |
| 17 2000 μM | N.A. | 0.5 IC50 value | FIG. 5 |

In addition, Table 6 below is a list of further experimental data obtained for compounds using the assay described above.

TABLE 6
average AC50 values in μM, n = 6 for compounds 3, 18, 19,23, 28, 29.

| Compound name & No. | AC50 μM |
|---|---|
| 2-N(2,4-dihydroxy-phenyl)pyridine-2,5-dicarboxamid, 3 | 10 |
| 2-N(3-hydroxy-2-naphthyl)pyridine-2,5-dicarboxamid, 18 | 115.8 |
| 2-N(1-hydroxy-2-naphthyl)pyridine-2,5-dicarboxamid, 19 | 31.6 |
| 2-N(2-hydroxy-5-chloro-phenyl)pyridine-2,5-dicarboxamid, | >200 |
| 23 | |
| 2-N(2-hydroxy-3-methoxy-1-naphthyl)pyridine-2,5- | 174.6 |
| dicarboxamid, 28 | |
| 2-N(2-hydroxy-6-chloro-1-naphthyl)pyridine-2,5- | 113.2 |
| dicarboxamid, 29 | |

The data in Table 5 and 6 clearly shows that the compounds of the invention provide novel Sirt1 modulators and generally these are activators having activity in the micromolar range, while inhibition of Sirt1 is also obtained, e.g. comp. 17 having an IC50 of 0.5 μM.

The present invention provides among other things sirtuin-activating compounds and/or NAD mimicking compounds and methods of use thereof. The compounds of formulae I, II, III and IV are useful in a medical composition comprising an effective amount of said compound and a pharmaceutically acceptable carrier. Said medical composition is useful in a method for treating a patient suffering from a sirtuin mediated disorder comprising administering to said patient an effective amount of said medical composition. While specific embodiments of the subject invention have been discussed, the above specification is illustrative and not restrictive.

### Example 4

### Absorption, Distribution, Metabolism, Excretion (ADME) Profiling of SIRT1 Activators

Six SIRT 1 activators were profiled in selected physical property and ADME assays in order to assess the potential pharmacokinetic properties of these key compounds.

Physical Properties

Physical properties tPSA and ClogP (calculated lipophilicity octanol/water) were calculated using ChemDraw. Additionally, M log D_{7.4} (measured partition coefficient at pH 7.4) and log P₁₁ (measured partition coefficient at pH 11) were measured, and all data are shown in Table 7.

TABLE 7

| | Compound | | | | | |
|---|---|---|---|---|---|---|
| Property | 3 | 9 | 11 | 19 | 24 | 29 |
| tPSA | 125 | 125 | 105 | 105 | 114 | 105 |
| (A²) | | | | | | |
| ClogP | 0.3 | 0.3 | 1.9 | 2.1 | 0.8 | 3.1 |
| MlogD_{7.4} | 0.8 | 0.3 | 1.6 | ND | ND | ND |
| MlogP₁₁ | 1.0 | NA | −0.1 | ND | ND | ND |

The calculated tPSA (Topological polar surface area) values are between 105-125 and the ClogP values 0.3-3.1 (Table 7). Measured log D_{7.4} (lipophilicity at pH 7.4) values for the compounds assessed experimentally agreed with the C log P values. M log P₁₁ values for example compound 3 agreed with other lipophilicity values shown. In conclusion the data listed in Table 7 clearly demonstrates that the compounds of the invention are likely drug candidates.

### Kinetic Aqueous Solubility

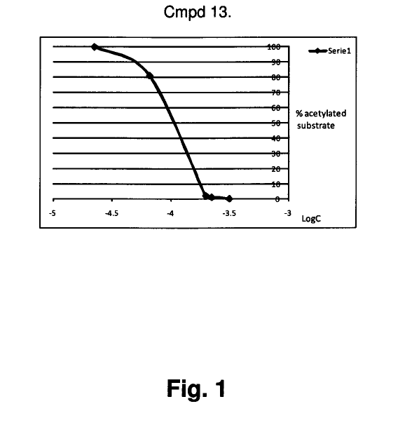

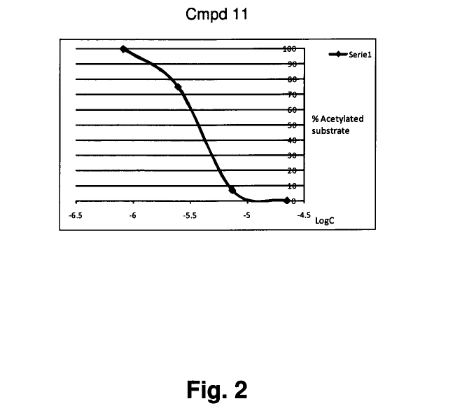

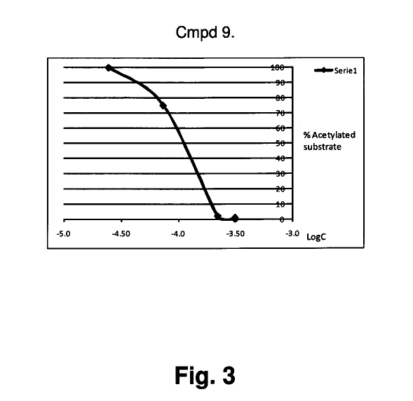

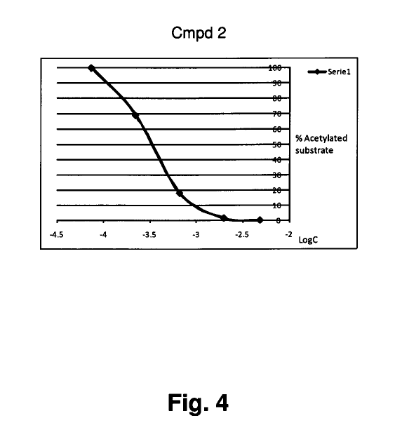

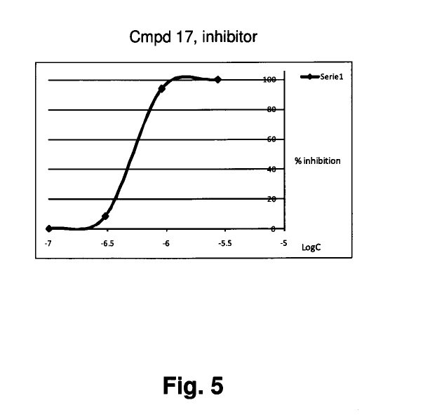

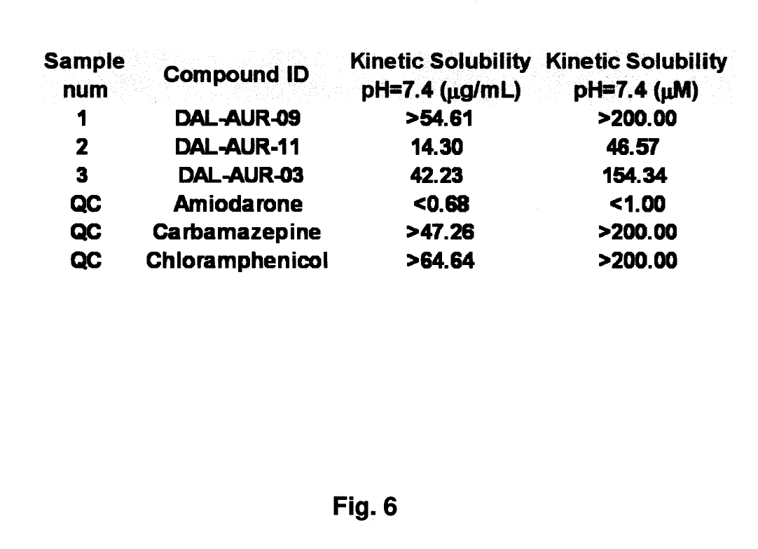

Measured kinetic solubility of representative compounds 3, 9, and 11 were between 14 and 55 μg/mL (FIG. 6). In conclusion the data in FIG. 6 demonstrates that the compounds of the invention from a solubility point of view are likely drug candidates.

### Metabolic Stability in Hepatocytes

Stability of the six compounds in the presence of a drug metabolizing system such as human and rat hepatocytes was measured. Data were reported from the CRO in the format of hepatic intrinsic clearance (CLint) in mL/min/kg. These CLint data were converted using the well-stirred equation

CLh=CLint×Qh/CLint+Qh

to a whole body clearance (CLh, hepatic clearance), which is more directly physiologically interpreted (Qh, hepatic blood flow rate, mL/min/kg). The data are shown in Table 8.

TABLE 8

| | mL/min/kg | | | mL/min/kg | | |
|---|---|---|---|---|---|---|
| | rat | rat | | hu | hu | |
| Compound | CLint | CLh | % Qh | CLint | CLh | % Qh |
| 9 | 15 | 11.8 | 21.4 | 12 | 7.6 | 36.4 |
| 11 | 133 | 38.9 | 70.7 | 32 | 12.7 | 60.4 |
| 3 | 53 | 27.0 | 49.1 | 83 | 16.8 | 79.8 |
| 7EC | 107 | 36.3 | 66.0 | 99 | 17.3 | 82.5 |
| 7HC | 255 | 45.2 | 82.3 | 171 | 18.7 | 89.1 |

Italics = <(less than) values
rat Qh = 55 mL/min/kg
human Qh = 21 mL/min/kg

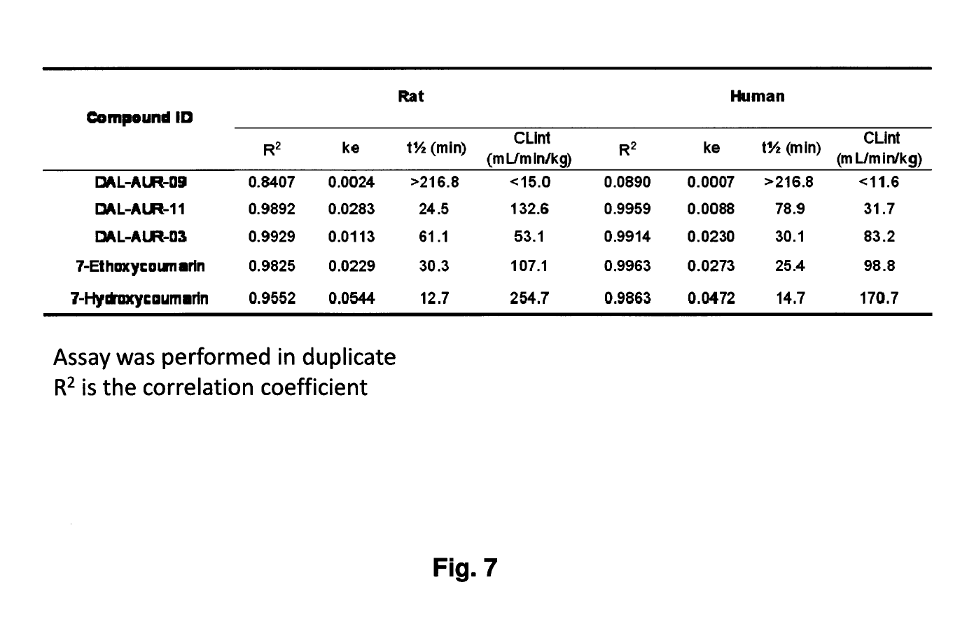

Compounds 3, 9, and 11 show relatively low clearance (FIG. 7), and good stability in this system. In conclusion the data shown in table 8 clearly demonstrate that the compounds of the invention are likely drug candidates.

### Plasma and Brain Homogenate Binding

These types of binding data can be used separately to understand free drug available in blood and brain, respectively, which could be available for target engagement, distribution to other tissues, or elimination. Together, however, these parameters can be used to estimate the expected brain/plasma ratio based on free drug distribution in the absence of active transport (e.g. P-gp mediated efflux).

[free plasma]=[free brain]

fuₚₗ×[plasma]=fu_{br}×[brain]

fuₚₗ/fu_{br}=[brain]/[plasma]
where fu=fraction unbound, and pl and br are plasma and brain, respectively.

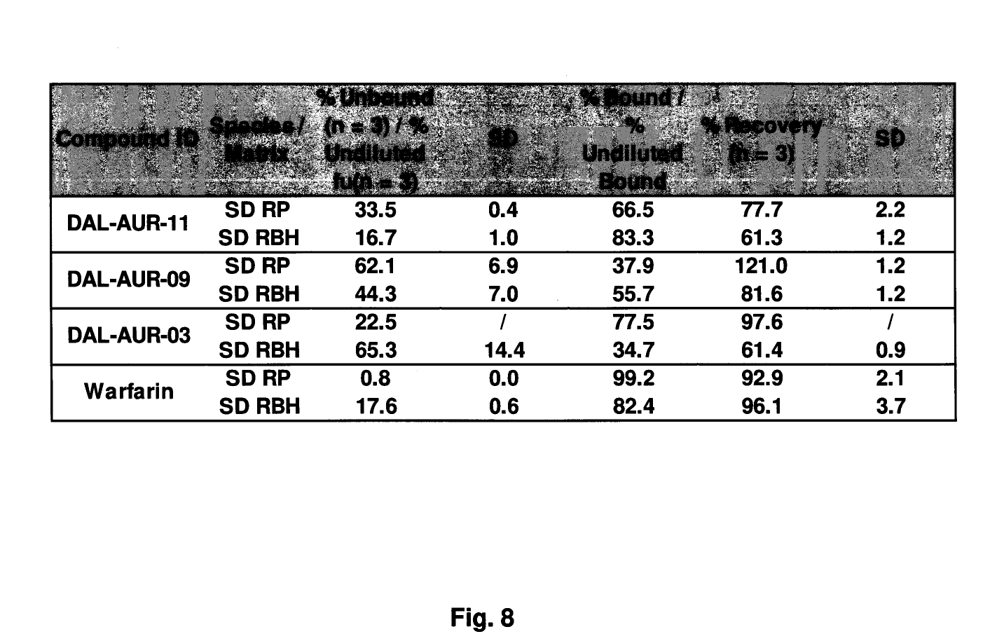

These data show that the compounds of the invention demonstrate in vitro plasma/brain free fraction ratios giving in vivo brain/plasma ratios >1(FIG. 8). In conclusion these data show that the brain penetration of compounds of the invention are to be significant.

### Efflux Ratio Determination in MDCK-MDR1 Cells

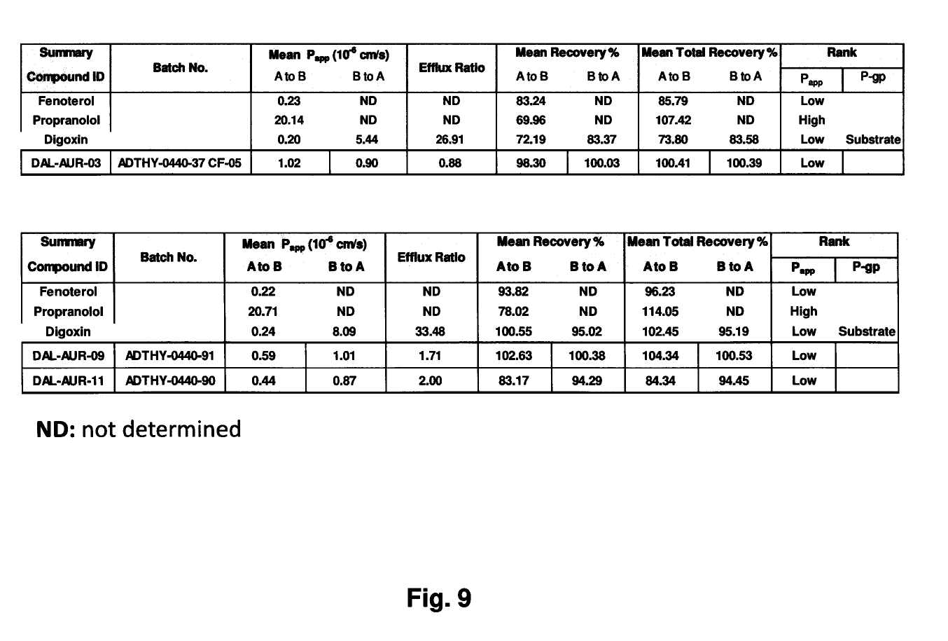

MDR1 is the nomenclature for the major efflux transporter P-glycoprotein (P-gp), which is expressed at important anatomical barriers such as blood-brain, small intestine, and tumor cells. The MDCK-MDR model is a polarized cell model over expressing P-gp (i.e. P-gp is expressed on the Apical, or gut lumen, side of the cell) that is capable of actively effluxing small molecule substrates in the B to A direction. This system measures passive permeability through a cell (Papp), and also the propensity to be actively effluxed by P-gp, which would appear as a higher B→A flux than A→B. For example, the positive control for active P-gp efflux, digoxin, shows an A→B<0.25, a B→A>5, and an efflux ratio >26. The compounds were run bidirectionally (Compound added to apical side and flux measured of compound appearing on the basolateral, or blood, side; compound added on basolateral side and flux of compound to apical side measured). Permeability data (FIG. 9) for the compounds 3, 9 and 11 clearly demonstrates moderate cell permeability, and no P-gp mediated efflux. In conclusion the data show that the compounds of the invention will have moderate permeability across cell barriers with no efflux issues due to P-gp.

### CYP Inhibition

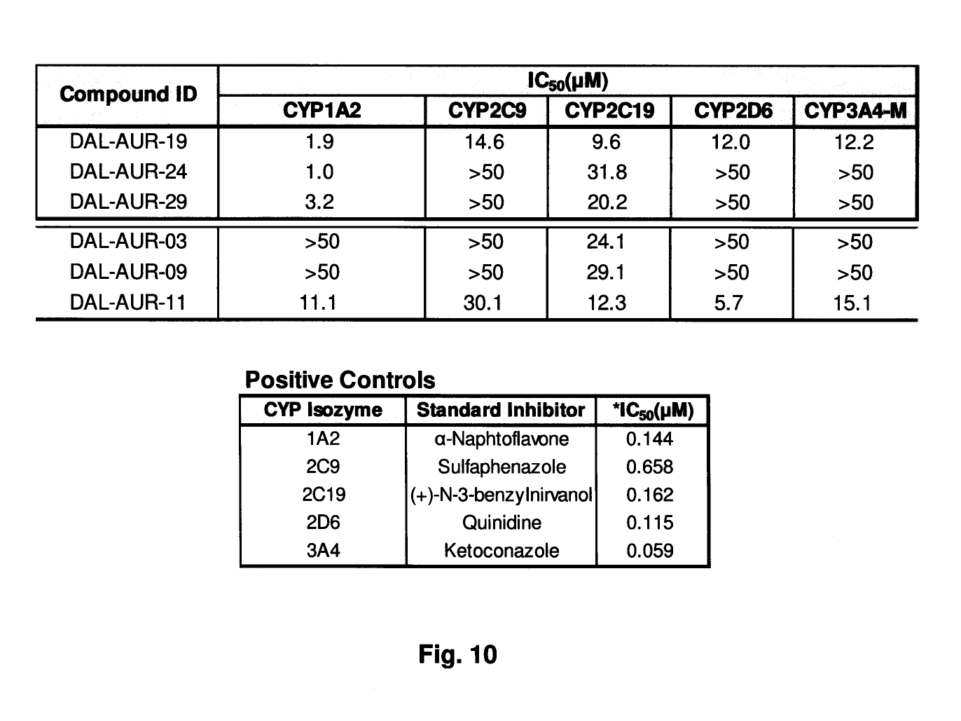

Inhibition of cytochrome P450 enzymes (CYP) is a common mechanism for potentially significant DDI (drug-drug interactions) to occur. In vitro assays assessing the propensity for molecules to inhibit CYP enzymes are often used to attempt to filter out or de-risk molecules. The compounds 3, 9, 11, 19, 24, and 29 were assessed for their inhibition potency toward CYP1A2, 2C9, 2C19, 2D6, and 3A4 (FIG. 10). The compounds showed relatively weak (>5 mM) or no potency toward these CYPs. In conclusion the compounds of the invention do not show any significant inhibition of the major drug metabolizing CYPs.

Effect of Solubility/Permeability/Metabolism Data on Absorption and Penetration into Cells

The propensity for a compound to have adequate oral absorption through the intestine is a combination of aqueous solubility, membrane permeability, minimal efflux, and minimal first-pass hepatic metabolism. The compounds of the invention show moderate permeability with no efflux, and good solubility. This combined with the observed low hepatic metabolism should accordingly result in adequate oral bioavailability in humans.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All devices and methods that embody the present invention can, in alternate embodiments, be more specifically defined by any of the transitional terms “comprising”, “consisting essentially of” and “consisting of”.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference are the following PCT publications: WO 2005002672; 2005002555; and 2004016726.

### REFERENCES

P. Onyango et al., Proc. Natl. Acad. Sci. USA 99: 13653-13658 (2002)). SIRT3 is a homolog of SIRT1 that is conserved in prokaryotes and eukaryotes.
B. Schwer et al., J. Cell Biol. 158: 647-657 (2002)). SIRT3 is believed to be cleaved into a smaller, active form by a mitochondrial matrix processing peptidase (MPP).
Masoro, E. J. Experimental Gerontology 35 (2000) 299-305) Caloric restriction and aging: an update.
T. J. Lin et al. (Journal of Cataract & Refractive Surgery, Volume 37, Issue 7, July 2011, Pages 1270-1274).
Hoff, K. G., Avalos, J. L., Sens, K., Wolberger, C. (2006) Insights into the sirtuin mechanism from ternary complexes containing NAD+ and acetylated peptide. Structure 14: 1231-1240.
Cosgrove, M. S. On the Structural Basis of Sirtuin Substrate Affinity (2006) Biochemistry 45: 7511-7521.
Avalos, J. L., Bever, K. M., Wolberger, C. Mechanism of sirtuin inhibition by nicotinamide: altering the NAD(+) cosubstrate specificity of a Sir2 enzyme (2005) Mol. Cell 17: 855-868.
Fan Yeung Jamie E Hoberg, Catherine S Ramsey, Michael D Keller, David R Jones, Roy A Frye and Marty W Mayo. Modulation of NF-κB-dependent transcription and cell survival by the SIRT1 deacetylase. The EMBO Journal (2004) 23, 2369-2380.
Pacholec M, Chrunyk B A, Cunningham D, Flynn D, Griffith D A, Griffor M, Loulakis P, Pabst B, Qiu X, Stockman B, Thanabal V, Varghese A, Ward J, Withka J, Ahn K. SRT1720, SRT2183, SRT1460, and resveratrol are not direct activators of SIRT1. J Biol Chem. 2010.
Hashimoto et al. (2010), Biogerontology Volume 11, Number 1, 31-43.
R. Larock, Comprehensive Organic Transformations (1989)
T. W. Greene and P. G. M. Wuts, Protective Groups in Organic Synthesis, 2d. Ed. (1991)
L. Fieser and M. Fieser, Fieser and Fieser's Reagents for Organic Synthesis (1994)
L. Paquette, ed., Encyclopedia of Reagents for Organic Synthesis (1995).
Blum C A, Ellis J L, Loh C, Ng P Y, Perni R B, Stein R L. SIRT1 modulation as a novel approach to the treatment of diseases of aging. J Med Chem. 2011 Jan. 27; 54(2):417-32.
Pfister J A, Ma C, Morrison B E, D'Mello S R (2008) (Opposing Effects of Sirtuins on Neuronal Survival: SIRT1-Mediated Neuroprotection Is Independent of Its Deacetylase Activity. PLoS ONE 3(12): e4090. doi:10.1371/journal.pone.0004090).
Robers M B, Loh C, Carlson C B, Yang H, Frey E A, Hermanson S B, Bi K. Measurement of the cellular deacetylase activity of SIRT1 on p53 via LanthaScreen® technology. Mol Biosyst. 2011; 7(1):59-66.
Walker A K, Yang F, Jiang K, Ji J Y, Watts J L, Purushotham A, Boss O, Hirsch M L, Ribich S, Smith J J, Israelian K, Westphal C H, Rodgers J T, Shioda T, Elson S L, Mulligan P, Najafi-Shoushtari H, Black J C, Thakur J K, Kadyk L C, Whetstine J R, Mostoslaysky R, Puigserver P, Li X, Dyson N J, Hart A C, Naar A M. Conserved role of SIRT1 orthologs in fasting-dependent inhibition of the lipid/cholesterol regulator SREBP. Genes Dev. 2010 Jul. 1; 24(13):1403-17.
Rutanen J, Yaluri N, Modi S, Pihlajamäki J, Vänttinen M, Itkonen P, Kainulainen S, Yamamoto H, Lagouge M, Sinclair D A, Elliott P, Westphal C, Auwerx J, Laakso M. SIRT1 mRNA expression may be associated with energy expenditure and insulin sensitivity. Diabetes. 2010 April; 59(4):829-35. Epub 2010 Jan. 27. PMID: 20107110
Dai H, Kustigian L, Carney D, Case A., Considine T, Hubbard B, Perni R, Riera T, Szczepankiewicz B, Vlasuk G, Stein R. SIRT1 activation by small molecules—kinetic and biophysical evidence for direct interaction of enzyme and activator. J Biol Chem. 2010 Oct. 22; 285(43):32695-703. Epub 2010 Aug. 11. PMID: 20702418
Yoshizaki T, Schenk S, Imamura T, Babendure J L, Sonoda N, Bae E J, Oh da Y, Lu M, Milne J C, Westphal C, Bandyopadhyay G, Olefsky J M. SIRT1 inhibits inflammatory pathways in macrophages and modulates insulin sensitivity. Am J Physiol Endocrinol Metab. 2010; 298(3):E419-428. PMID: 19996381.
Pillarisetti, S. Recent Patents in Cardiovascular Drug Discovery, 2008, 3, 315-64.

1. A compound of Formula I 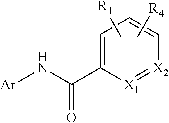
wherein Ar is a C₆-C₁₄ carbocyclic or heterocyclic aromatic ring, said Ar group being substituted with at least one substituent R2 selected from the group of OH, and SH, and further optionally substituted with at least one substituent R3 selected from the group of cyano, NO₂, a halogen, such as F, Cl, Br, —CONRxRy, —NHCOAlkyl, —COOH, —COOAlkyl, —SOAlkyl, —SO₂Alkyl,
—NRxRyRz, OH, —NRxRy, SH, Alkoxy, thioalkoxy, alkyl and aryl, wherein Rx, Ry and Rz represent an alkyl group, and the alkyl and alkoxy moieties are preferably of lower carbon chain lengths, such as C₁₋₄, and the aryl moiety is preferably a phenyl group;
X₁═N or C,
X₂═N or C, and X₁≠X₂, or one of X₁ and X₂ is absent resulting in a pyrrol ring,
R1 is a substituent selected from —C(═O)—R5, wherein R5 is NH₂ optionally mono- or di-substituted with C₁₋₄ lower alkyl such as CH₃;
R4 is an optional substituent selected from lower alkyl such as methyl;
and a solvate, tautomer or isomer thereof including pharmaceutically acceptable salts, acid addition salts and base addition salts.

2. The compound of claim 1, wherein Ar is naphthyl or phenyl.

3. A compound according to claim 1 wherein
Ar is a phenyl ring having a hydroxy substituent in the 2 position, R1 is —C(═O)—NH₂, R4 is H; and a solvate, tautomer or isomer thereof including pharmaceutically acceptable salts, acid addition salts and base addition salts.

4. A compound of claim 2 selected from
2-N(2-hydroxyphenyl)pyridine-2,5-dicarboxamide,
5-N(2,4-dihydroxyphenyl)pyridine-2,5-dicarboxamide,
2-N(2,4-dihydroxyphenyl)pyridine-2,6-dicarboxamide,
5-N(2-hydroxyphenyl)pyridine-2,5-dicarboxamide, and a solvate, tautomer or isomer thereof including pharmaceutically acceptable salts, acid addition salts and base addition salts.

5. A compound of claim 3 wherein Ar is further substituted in the 3 or 5 position with a halogen substituent, such as fluoro, chloro, or bromo; or the additional substituent is a lower alkoxy such as methoxy.

6. A compound of claim 5 selected from 2-N(2-hydroxy-5-chloro-phenyl)pyridine-2,5-dicarboxamide, and a solvate, tautomer or isomer thereof including pharmaceutically acceptable salts, acid addition salts and base addition salts.

7. A compound of claim 1 wherein one of X₁ and X₂ is absent resulting in a pyrrol ring, such as the compound 4-N(2-hydroxyphenyl)pyrrol-2,4-dicarboxamide, and a solvate, tautomer or isomer thereof including pharmaceutically acceptable salts, acid addition salts and base addition salts.

8. A compound of claim 1 wherein
Ar is a naphthyl ring having a hydroxy substituent preferably in the 2, 3, 6 or 8 position, R1 is —C(═O)—NH₂;
and R4 is H; and a solvate, tautomer or isomer thereof including pharmaceutically acceptable salts, acid addition salts and base addition salts.

9. A compound of claim 8 wherein Ar is further substituted in the 3 or 6 position with a halogen substituent, such as fluoro, chloro or bromo; or the additional substituent is a lower alkoxy such as methoxy.

10. A compound of claim 8 selected from
2-N(6-hydroxy-1-naphthyl)pyridine-2,5-dicarboxamide,
5-N(5-hydroxy-1-naphthyl)pyridine-2,5-dicarboxamide,
5-N(6-hydroxy-1-naphthyl)pyridine-2,5-dicarboxamide,
5-N(4-hydroxy-1-naphthyl)pyridine-2,5-dicarboxamide,
and a solvate, tautomer or isomer thereof including pharmaceutically acceptable salts, acid addition salts and base addition salts.

11. A compound of claim 8 selected from
2-N(3-hydroxy-2-naphthyl)pyridine-2,5-dicarboxamide
2-N(1-hydroxy-2-naphthyl)pyridine-2,5-dicarboxamide, and a solvate, tautomer or isomer thereof including pharmaceutically acceptable salts, acid addition salts and base addition salts.

12. A compound of Formula Ia: 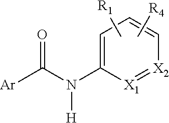
wherein all substituents are as defined in claim 1, and a solvate, tautomer or isomer thereof including pharmaceutically acceptable salts, acid addition salts and base addition salts.

13. A compound of claim 12 selected from
6-(2-hydroxy-benzoylamino)-pyridine-3-carboxamide and
6-(2-hydroxy-1-naphthoylamino)-pyridine-3-carboxamide, and a solvate, tautomer or isomer thereof including pharmaceutically acceptable salts, acid addition salts and base addition salts.

14. A medical composition comprising an effective amount of a compound according to claim 1 and a pharmaceutically acceptable carrier.

15. A method for treating a patient suffering from a sirtuin mediated disorder comprising administering to said patient an effective amount of a medical composition including a compound according to claim 1.

16. (canceled)

17. A method according to claim 15 wherein said sirtuin mediated disorder is alleviated through activation of the deacetylating activity of said sirtuin.

18. A method according to claim 15 wherein said sirtuin mediated disorder is alleviated through inhibition of the deacetylating activity of said sirtuin.

19. A method for treating a patient suffering from a nicotinamide adenine dinucleotide deficiency disorder comprising administering to said patient an effective amount of a medical composition including a compound according to claim 1.

20. (canceled)

21. A compound of claim 9 selected from
2-N(2-hydroxy-3-methoxy-1-naphthyl)pyridine-2,5-dicarboxamide
2-N(2-hydroxy-6-chloro-1-naphthyl)pyridine-2,5-dicarboxamide, and a solvate, tautomer or isomer thereof including pharmaceutically acceptable salts, acid addition salts and base addition salts.
